(19) United States
(12) Patent Application Publication
(10) Pub. No.: US 2005/0124515 A1
(43) Pub. Date: Jun. 9, 2005

(54) Soap bar compositions comprising alpha sulfonated fatty acid alkyl estersand polyhydridic alcohols and process for producing same

(76) Inventors: Carlos E Ospinal, Streamwood, IL (US); Jeffrey S Nelson, Lake Bluff, IL (US); Matthew I Levinson, Lincolnwood, IL (US); Catherine J. Sporer, Lindenhurst, IL (US); Rao Kameshwer, Skokie, IL (US); Xue Min Dong, Lincolnshire, IL (US)

Correspondence Address: MCANDREWS HELD & MALLOY, LTD, 500 WEST MADISON STREET, SUITE 3400, CHICAGO, IL, 60661, US

(21) Appl. No.: 10/502,915

(22) PCT Filed: Jan. 31, 2003
(86) PCT No.: PCT/US03/02861

Related U.S. Application Data
(60) Provisional application No. 60/353,693, filed on Jan. 31, 2002.

(51) Int. Cl. A61K 7/50

(52) U.S. Cl. 510/156

(57) ABSTRACT

Disclosed are improved cleaning compositions comprising soap&com ma; fatty acid, synthetic detersive surfactant, salt and a polyhydridic alcohol, which are suitable for formation into precursor cleansing/laundry bar “soap noodles,” personal cleansing bars and laundry detergent bars. The compositions comprise: (a) from about 58% to about 93% by weight of an approximately 70% aqueous soap slurry; (b) from about 1% to about 15% by weight of a fatty acid; (c) from about 2% to about 30% by weight of an approximately 55% aqueous mixture of anionic surfactants comprising i) an alpha sulfonated alkyl ester, and ii) a sulfonated fatty acid or salts thereof, wherein the ratio of i) to ii) is from about 10:1 to about 1:10; (d) from about 0.5% to about 2% by weight of a salt; (e) from about 0.5% to about 5% by weight of a 25 polyhydridic alcohol; and (f) from 0 to about 10% by weight of an alkanolamide of the formula O CH₃(CH2)ₙCNH(CH₂)_{y}OH wherein n=6-16, and y is 2-4; The invention additionally relates to an improved process for producing both precursor cleansing/laundry bar “soap noodles” and personal cleansing/laundry detergent bars 35 comprising combining (a)-(e) to form a liquid mixture at a temperature of about 65° C. to about 105° C., removing from about 50% to about 90% by weight of the water from the liquid mixture, by heating up to 150° C. under vacuum conditions or 105° C. at normal conditions to form a thickened mixture, extruding the thickened mixture to form flaked solid or semi-solid pellets or noodles, and optionally, plodding the flaked solid or semi-solid pellets or noodles to form plodded pellets or noodles,f extruding the plodded pellets or noodles to form a billet, cutting the billet, and stamping the cut billet to yield a personal cleansing/laundry detergent bar.

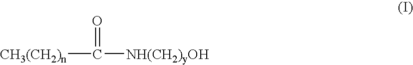

### CROSS REFERENCE TO RELATED APPLICATIONS

This application claims priority from U.S. Provisional Application Ser. No. 60/353,693, filed Jan. 31, 2002, the disclosure of which is incorporated herein by reference in its entirety.

### BACKGROUND OF TEE INVENTION

1. Field of the Invention

This invention relates to cleaning compositions comprising a soap, a fatty acid, a synthetic detersive surfactant, a salt and a polyhydridic alcohol, wherein said compositions are suitable for formation into precursor cleansing/laundry bar surfactant pre-blends (i.e., “soap noodles”), personal cleansing bars and laundry detergent bars. Specifically, the invention relates to liquid, paste, and flaked compositions containing α-sulfonated fatty acid alkyl esters which are suitable for processing into solid or semi-solid personal cleansing bars and laundry detergent bars. The instant invention additionally relates to an improved process for producing both precursor cleansing/laundry bar surfactant pre-blends/“soap noodles” and personal cleansing/laundry detergent bars which contain α-sulfonated fatty acid alkyl esters. The inventive compositions possess improved processing characteristics and allow for formation of bars which exhibit improved hardness, improved resistance to marring, lowered wear-rate and decreased mush formation during consumer use.

2. Description of the Related Art

Personal cleansing and laundry cleaning bars, and their precursor formulations, have become a focus of great interest. People generally wash and exfoliate their skin with various surface-active detergent bar formulations several times a day. Ideal skin cleanser bars should cleanse the skin gently, causing little or no irritation, without de-fatting and over-drying the skin or leaving it taut after frequent routine use. Most high lathering soap bars fail in this respect.

The processability, firmness, smearing and marring properties of personal cleansing and laundry cleaning bars and the processability of their precursor detergent compositions has become a focus of great interest. Precursor cleansing/laundry bar surfactant pre-blends which have lowered viscosities and are easily extruded and plodded are highly desirable. Final bars which are easily processed from such precursor compositions which are also very mild, firm but not hard, have low smear and do not readily mar are also highly desirable.

Synthetic detergent bars, frequently called “combo bars.” (i.e., a bar having substantial amounts of soap) and/or “syndet bars” (i.e., a bar having very little or no soap) are well known to the art, along with natural “soap” bars for personal care use. Syndet bars often possess poor physical properties, e.g., off odors, poor processability, stickiness, brittleness, bar mushiness, poor lather quality, lack of mildness or combinations thereof. Additionally, the problems of formulating synthetic detergent bars are not limited to the performance characteristics of the finished bars. Most synthetic bars which are made with certain mild surfactants are very difficult to fabricate. Processing conditions for such bars present relatively high technical challenges to commercial scale manufacturers, due primarily to the need of expensive special handling equipment.

In contrast, the fabrication of relatively pure “soap” bars is a well-worked-out engineering procedure involving milling, plodding and molding. For example, coco/tallow soap becomes quite plastic when warmed and can be easily plodded and molded under relatively low pressures. However, most synthetic detergents and detergent-filler compositions for use in cleansing or laundry detergent bars become overly plastic and pasty and the machinery for fabrication and processing is often complicated and must be specially designed. See, e.g., U.S. Pat. No. 2,678,921, issued May 18, 1954. Ideally, processing of syndet bars or synthetic detergent bars should be fast and problem free in terms of milling, extruding, plodding, molding and stamping the finished bar formation. Most mild syndet bar processings fall short in some or all of these respects.

Synthetic detergent bar formulations for personal care use are well known to the art. For example, see U.S. Pat. No. 5,328,632, issued Jul. 12, 1994; U.S. Pat. No. 5,510,050, issued Apr. 23, 1996; U.S. Pat. No. 5,393,449, issued Feb. 28, 1995; WO 95/27036, filed Mar. 30, 1995; and WO 95/27038, filed Mar. 30, 1995. The major drawbacks of most synthetic surfactant toilet bar formulations include poor lather, poor smear, and poor processability due to stickiness. The use of high sudsing anionic surfactants can yield acceptable lather volume, but unfortunately, the use of high sudsing anionic surfactants does, in fact, lead to poor processability. While some known mild blends of sodium coconut/tallow alkyl glyceryl ether sulfonate (AGS) are relatively good in lather potential, they are difficult to process because of their stickiness or hygroscopicity. It will be appreciated that processability, firmness, smear, low marring, mildness, lather, and rinsability make surfactant selection and stoichiometry of ingredients for mild personal cleansing bars a critical and difficult task. Thus, it will also be appreciated that rather stringent requirements for formulating mild personal cleansing bars limit the choice of surfactants, and final formulations represent some degree of compromise. Mildness is often obtained at the expense of processability, effective cleansing, lathering, or rinsing, or vice versa. Processability is often obtained at the expense of smear or marring of the finished bar.

Synthetic detergent bar formulations for laundry cleaning are also well known to the art. For example, see U.S. Pat. No. 5,965,508, issued Oct. 12, 1999; WO 95/27036, filed Mar. 30, 1995; and WO 95/27038, filed Mar. 30, 1995. Such laundry detergent bars have found expanded use in regions of the world where automatic clothes washing machines are not common. The ideal laundry detergent bar is effective in cleaning clothes, has acceptable sudsing characteristics, low smear, and pleasing odor and appearance. As these laundry detergent bars are in contact with the skin during clothes washing, mildness is also highly desirable.

Methods for making laundry detergent bars are well known in the art. For example, see Philippine Pat. No. 23,689, issued Sep. 27, 1989; and Philippine Pat. No. 24,551, issued Aug. 3, 1990. Much like the syndet bars for personal care use, laundry detergent bars often possess many of the same physiochemical problems, e.g., harshness, poor lather, poor smear, poor marring and poor processability due to stickiness.

Conventionally milled toilet soaps are made by a process which comprises (1) drying soap having a moisture content of from about 28% to about 30% down to a moisture content of about 7% to about 14%, (2) forming the dried soap into precursor “soap noodles,” by passing it through a plodder, (3) mixing the various desired additives such as colorants, perfume, etc., into the soap noodles, (4) passing the mixture formed in (3) through a mill or series of mills (“milling” the soap) thereby forming ribbons of soap, (5) passing the milled soap mixture from (5) through a plodder to form a log of soap (i.e., “plodding” the soap to form a billet), and (6) cutting the log into segments (i.e., billets) and stamping the segments into the desired bar shape.

The soap which is dried in step (1) can be made from saponification of fats or neutralization of free fatty acids. Because the drying is never completely uniform, the dried soap inevitably contains some particles which are over-dried and are harder than the remaining bulk of the dried soap. If the soap also contains free fatty acid, non-homogeneity of the free acid in the soap can also contribute to the presence of soap particles which are harder than the remaining bulk of the dried soap. The hard particles are from about 0.5 to about 10 mm in diameter. These particles remain in the soap through the first plodding step (2) and the mixing step (3). In the milling step (4), the soap is “worked” and the over-dried particles are broken down into much smaller particles (generally less than about 0.25 mm in diameter) and are homogeneously distributed throughout the soap mass. In the absence of milling, the finished bar may exhibit a rough or sandy feel during use, due to the slower dissolution rate of the relatively large over-dried soap particles, also called “hard specks.” When the soap has been properly milled, the over-dried soap cannot be detected during use, because it has been reduced to a much smaller particle size and is distributed uniformly throughout the soap mass. See British Pat. No. 512,551, issued Sep. 19, 1939, incorporated herein by reference (from U.S. Pat. No. 4,405,492).

Mild, detergent-soap, toilet bars containing C₆-C₁₈ acyl isethionate as the principal detergent and minor amounts of fatty acids and soap are disclosed in U.S. Pat. No. 2,894,912 ('912 patent) and U.S. Pat. No. 3,376,229 ('229 patent). In the '912 patent, the chips processed into bars are produced from either a 40-50% aqueous slurry of the ingredients mixed at a temperature of from 38° C. to 93° C. or a mixture of the dry ingredients mixed at 100° C. for a long period of time. In the '229 patent, the bars are prepared from a liquid mixture of acyl isethionate, fatty acids, anionic syudet and soap mixed at a temperature of about 110° C. to 113° C. for about fifteen minutes. The latter bars contain at least about 4% by weight of sodium isethionate as a processing aid.

In U.S. Pat. No. 4,707,288, mixtures of acyl isethionate, fatty acids, soap and more than 2% by weight of sodium isethionate are mixed in particulate form at temperatures in the range of 60° C. to 86° C. using a special cavity transfer mixer under conditions of high shear to yield toilet bars which exhibit reduced grit.

U.S. Pat. No. 4,696,767, discloses a process for making mild toilet bars wherein a slurry of acyl isethionate, water and a polyol such as sorbitol is formed into a stable solution by heating at a temperature of from 100° C. to 120° C. at 4-10 p.s.i.g. and said slurry is mixed with neat soap and this mixture is heated to about 150° C. under a pressure of 4 atmospheres before being spread through a vacuum drying and plodding step to provide flakes which yield a toilet bar without grit. However, the presence of the polyol leads to increased water penetration in the soap dish as well as a bar of increased cost. This patent further teaches that use of acyl isethionate in particulate form causes problems—fine particles function as a lacrimatory agent (i.e. there is weeping of material out of the soap bar) and larger particles yield bars with grit.

In U.S. Pat. No. 4,663,070, a toilet bar composition in which soap is the principal surfactant is described. Liquid mixtures containing a major proportion of soap plus acyl isethionate, fatty acids, water and sodium isethionate were formed at temperatures of 96° C. to 103° C. In U.S. Pat. No. 5,030,376, a similar mixture containing a major proportion of soap is processed under conditions of high shear in a special cavity transfer mixer at temperatures maintained below 40° C. to form a mixture with some of the soap in the delta phase. U.S. Pat. No. 5,041,233, also relates to a similar mixture wherein a mixture of acyl isethionate, fatty acids and soap is prepared at a temperature of 82° C. to 94° C., with the soap being formed in situ. This patent indicates that high viscosity mixtures and hydrolysis of acyl isethionate can be problems in such mixtures.

The foregoing description of the relevant art indicates that a variety of processes have been employed to produce personal cleansing and laundry detergent bar pre-bends and the resulting mild, detergent-soap, toilet bars. Further, soap bars are commercially manufactured in a variety of aesthetically pleasing configurations. These products are frequently damaged by marring which is defined as the formation of undesirable, white, chalk-like shatter marks in and around dented areas on conventional soaps. Marring typically occurs during handling, shipping and distribution of finished products to customers.

Approximately one to two weeks after soap bar preparation, ordinary gift and decorative soaps bruise and chip especially on the edges and corners of intricate or unique configurations. When soap products are packed side-by-side, marring often occurs because individual bars bump against each other or against carton partitions and side walls. This marring is readily noticed, especially with colored soap where the chalk-like marks form around the bruises and chips.

Labor intensive packaging processes are currently used to protect conventional soap bases against marring. Novelty products which depend heavily on aesthetically pleasing qualities have previously required expensive cartons and/or protective wrappings to prevent surface defects. Even with these extra precautions, there is no guarantee that conventional formulations will avoid surface defects.

Thus, based on the foregoing, a need exists for superior personal cleansing and/or laundry detergent bar formulations with good mildness, improved processability, smear, lather potential, rinsability and low marring characteristics.

### SUMMARY OF THE INVENTION

The invention provides surprising performance in soap bar compositions. The inventive compositions comprise an alpha sulfonated alkyl ester, a sulfonated fatty acid, a soap, a fatty acid, a salt, and a polyhydridic alcohol and small amounts of water. Certain aspects of the invention provide synergistic results between the composition material. Compositions of the invention are useful in the production of precursor cleansing/laundry bar surfactant pre-blends or “soap noodles,” personal cleansing bars and laundry detergent bars, wherein such compositions exhibit improved processability, increased foaming properties, decreased smear properties, decreased marring properties, improved color stability, and/or impart superior feel and after-feel properties to skin.

It has been surprisingly discovered that the use of a polyhydridic alcohol greatly facilitates and improves the production of precursor cleansing/laundry bar “soap noodles” and personal cleansing/laundry detergent bars prepared from such noodles. The bars contain very low moisture levels, thus improving bar hardness properties and lowering wear rates during use. The compositions of the instant invention exhibit lower processing viscosities, improved drying characteristics, and are substantially free of gritty feel caused by the presence of hard particles of soap (“hard specks”), as compared to traditional bar compositions which are substantially free of polyhydridic alcohols.

The invention provides compositions suitable for formation of precursor cleansing/laundry bar “soap noodles” (i.e., personal cleansing and laundry detergent bar pre-blends), personal cleansing bars and laundry detergent bars. The compositions are useful in preparing stamped, personal cleansing and/or laundry detergent bars which have improved processability, are mild to the skin, have improved smear and bar firmness properties, have good lathering properties and/or reduced marring properties. The compositions of the invention may also be utilized to produce dish washing pastes, gels and body washes, along with other uses. Additionally, the invention provides improved processes for manufacturing precursor cleansing/laundry bar “soap noodles,” personal cleansing bars and laundry detergent bars.

The compositions of the invention may take the form of flaked/pellet solids, pastes, liquids, gels, ringing gels, or G-phase concentrates, depending upon the amount of water incorporated therein. In some embodiments, the compositions of the invention are in the form of precursor cleansing/laundry bar “soap noodles,” personal cleansing bars and/or laundry detergent bars.

The compositions of the invention are suitable for formation into precursor cleansing/laundry bar “soap noodles” or surfactant pre-blends, personal cleansing bars and laundry detergent bars and comprise:
(a) from about 58% to about 93% by weight of an approximately 70% aqueous soap slurry, the soap being of the formula 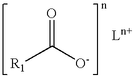
wherein R₁ is a C₆-C₂₂ hydrocarbyl group, an alkyl group, or combination thereof, n is 1 or 2, and L is a cation; and
(b) from about 1% to about 15% by weight of a fatty acid of the formula 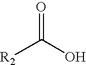
wherein R₂ is a C₆-C₂₂ hydrocarbyl group, an alkyl group, or combination thereof; and
(c) from about 2% to about 30% by weight of an approximately 55% aqueous mixture of anionic surfactants, the anionic surfactants comprising
   i) an alpha sulfonated alkyl ester of the formula 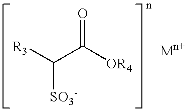
   wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
   ii) a sulfonated fatty acid of the formula 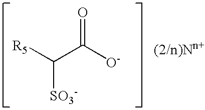
   wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
   wherein the ratio of i) to ii) is from about 10:1 to about 1:10;
(d) from about 0.5% to about 2% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof;
(e) from about 0.5% to about 5.0% by weight of a polyhydridic alcohol; and
(f) from 0 to about 10% by weight of an alkanolamide of the formula 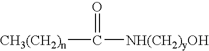
wherein n=6-16, and y is 2-4.

The inventive compositions have a reduced viscosity and are readily pumpable using standard soap bar production equipment, as compared to compositions prepared in the absence of said polyhydridic alcohol and salt. Additionally, the compositions of the invention are resistant to hydrolysis of the alpha sulfonated alkyl ester and/or the sulfonated fatty acid.

The compositions of the invention may be processed into precursor cleansing/laundry bar “soap noodles,” finished personal cleansing bars, laundry detergent bars, ordinary soap bars, “syndet” bars, or “combo” bars with the proper choice of optional components.

The compositions of the invention may be translucent and/or can also be processed into translucent personal cleansing and/or laundry detergent bars with the appropriate choice of additional components. The compositions are suitable for processing using standard extrusion and/or plodder equipment.

The invention further relates to an improved process to produce precursor cleansing/laundry bar “soap noodles,” personal cleansing bars and laundry detergent bars derived from the compositions of the invention. Accordingly, a process is provided for making personal cleansing and laundry detergent bar surfactant pre-blends or “soap noodles,” comprising the sequential steps of:
(a) forming at a temperature of about 65° C. to about 105° C. a substantially homogeneous aqueous liquid mixture comprising
   1. from about 58% to about 93% by weight of an approximately 70% aqueous soap slurry, the soap being of the formula 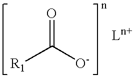
   wherein R₁ is a C₆-C₂₂ hydrocarbyl group, an alkyl group, or combination thereof, n is 1 or 2, and L is a cation; and
   2. from about 1% to about 15% by weight of a fatty acid of the formula 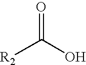
   wherein R₂ is a C₆-C₂₂ hydrocarbyl group, an alkyl group, or combination thereof; and
   3. from about 2% to about 30% by weight of an approximately 55% aqueous mixture of anionic surfactants, the anionic surfactants comprising:
      i) an alpha sulfonated alkyl ester of the formula 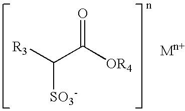
      wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
      ii) a sulfonated fatty acid of the formula 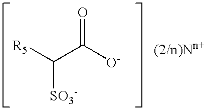
      wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
      wherein the ratio of i) to ii) is from about 10:1 to about 1:10;
   4. from about 0.5% to about 2% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof;
   5. from about 0.5% to about 10% by weight of a polyhydridic alcohol; and
   6. from 0 to about 10% by weight of an alkanolamide of the formula 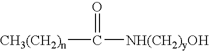
   wherein n=6-16, and y is 2-4;
(b) removing from about 5% to about 90% by weight of the total water from the liquid mixture to form a thickened mixture; and
(c) extruding the thickened mixture to form flaked solid or semi-solid particles.

This process may further comprise plodding the flaked solid or semi-solid particles to form plodded particles, extruding the plodded particles to form a billet, cutting the billet, and stamping the cut billet to yield a personal cleansing or laundry detergent bar.

The invention additionally encompasses bars which comprise the inventive compositions and bars produced by the processes described herein and processes to manufacture such bars.

### BRIEF DESCRIPTION OF THE DRAWINGS

FIG. 1 is a graph depicting continuous flow curves of SME soap slurries at 70° C. and constant shear rate of 2 l/s.

### DETAILED DESCRIPTION OF THE INVENTION

In one aspect, the invention relates to a process for preparing a personal cleansing and laundry detergent bar pre-blend, comprising the sequential steps of:
(a) forming at a temperature of about 65° C. to about 105° C. a substantially homogeneous aqueous liquid mixture comprising
   1. from about 58% to about 93% by weight of an approximately 70% aqueous soap slurry, the soap being of the formula 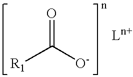
   wherein R₁ is a C₆-C₂₂ hydrocarbyl group, an alkyl group, or combination thereof, n is 1 or 2, and L is a cation; and
   2. from about 1% to about 15% by weight of a fatty acid of the formula 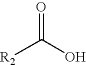
   wherein R₂ is a C₆-C₂₂ hydrocarbyl group, an alkyl group, or combination thereof; and
   3. from about 2% to about-30% by weight of an approximately 55% aqueous mixture of anionic surfactants, the anionic surfactants comprising:
      i) an alpha sulfonated alkyl ester of the formula 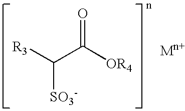
      wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
      ii) a sulfonated fatty acid of the formula 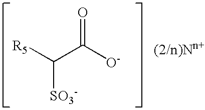
      wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
      wherein the ratio of i) to ii) is from about 10:1 to about 1:10;
   4. from about 0.5% to about 2% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof;
   5. from about 0.5% to about 10% by weight of a polyhydridic alcohol; and
   6. from 0 to about 10% by weight of an alkanolamide of the formula 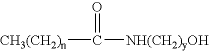
   wherein n=6-16, and y is 2-4;
(b) removing from about 5% to about 90% by weight of the total water from the liquid mixture to form a thickened mixture; and
(c) extruding the thickened mixture to form flaked solid or semi-solid particles.

This process embodiment may further comprise plodding the flaked solid or semi-solid particles to form plodded particles, extruding the plodded particles to form a billet, cutting the billet, and stamping the cut billet to yield a personal cleansing or laundry detergent bar. In accordance with this embodiment, preferably R₁ is a. C₆-C₁₈ hydrocarbyl group, an alkyl group, or combination thereof, and M is sodium or potassium, or a mixture thereof. Preferably, the soap is present from about 68% to about 78% by weight. Also preferably, R₂ is a C₁₂-C₂₀ hydrocarbyl group, an alkyl group, or combination thereof. More preferred fatty acids include coconut fatty acids and stearic acid and coconut fatty acid mixtures. Further in accordance with this process embodiment, the fatty acid is preferably present from about 2% to about 7% by weight. Also in a preferred embodiment, R₃ is a C₈-C₂₀ hydrocarbyl group, an alkyl group, or combination thereof, R₄ is methyl and M is hydrogen, sodium, potassium, calcium, magnesium ammonium, monoethanolammonium, diethanolammonium, triethanolammonium, or a mixture thereof; R₅ is a C₈-C₂₀ hydrocarbyl group, an alkyl group, or combination thereof, and N is hydrogen, sodium, potassium, calcium, magnesium, ammonium, monoethanolammonium, diethanolammonium, triethanolammonium, or a mixture thereof. Ideally, the ratio of the mixture of anionic surfactants is from about 3:1 to about 1:3. Further the preferred salt is sodium chloride. Also more preferably, the polyhydridic alcohol is selected from the group consisting of glycerine, polyglycerol esters, sorbitol and propylene glycol, or a mixture thereof; most preferably the polyhydridic alcohol is glycerine. Also preferably, y is 2. In accordance with this process embodiment, removing the water from the liquid mixture is accomplished by scraped wall vacuum evaporation drying under reduced pressure or heated drum drying at ambient pressure. Preferably, about 55% to about 85% by weight of the water is removed from the liquid mixture; and most preferably, about 60% to about 80 k by weight of the water is removed from the liquid mixture. The invention relates to a personal cleansing and laundry detergent bar pre-blend, produced by the process. Further in accordance with this embodiment, the invention relates to a personal cleansing and laundry detergent bar pre-blend, produced by the process and/or a personal cleansing and laundry bar produced by the process.

The inventive processes overcomes many of the shortcomings of the aforementioned heretofore known-processes. For example, the inventive process yields substantially homogeneous soap noodles which results in bars with minimal grit. Also, the process is carried out at temperatures at or below 105° C. so as to conserve energy and minimize hydrolysis of the alpha sulfonated alkyl ester. Additionally, the process utilizes standard bar processing equipment. Additionally, the bars resulting from the improved process have the desired hardness, water permeability, low grit and enhanced slip, and an absence of marring, even when dried to exceptionally low moisture levels, and with aging on the shelf for several months.

In another aspect, the invention relates to a process for preparing a personal cleansing and laundry detergent bar pre-blend, comprising the sequential steps of:
(a) forming at a temperature of about 65° C. to about 105° C. a substantially homogeneous aqueous soap-fatty acid liquid mixture comprising
   1. from about 58% to about 93% by weight of an approximately 70% aqueous soap slurry, the soap being of the formula 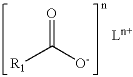
   wherein R₁ is a C₆-C₂₂ hydrocarbyl group, an alkyl group, or combination thereof, n is 1 or 2, and L is a cation; and
   2. from about 1% to about 15% by weight of a fatty acid of the formula 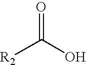
   wherein R₂ is a C₆-C₂₂ hydrocarbyl group, an alkyl group, or combination thereof; and
(b) adding to the soap-fatty acid liquid mixture to form a first intermediate liquid mixture at a temperature of about 65° C. to about 105° C.
   1. from about 0.5% to about 2% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof;
   2. from about 0.5% to about 5.0% by weight of a polyhydridic alcohol; and
   3. from 0 to about 10% by weight of an alkanolamide of the formula 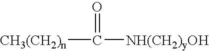
   wherein n=6-16, and y is 2-4;
(c) adding to the first intermediate liquid mixture to form a second intermediate liquid mixture at a temperature of about 65° C. to about 105° C. from about 2% to about 30% by weight of an approximately 55% aqueous mixture of anionic surfactants, the anionic surfactants comprising
   i) an alpha sulfonated alkyl ester of the formula 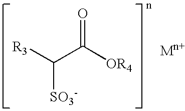
   wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
   ii) a sulfonated fatty acid of the formula 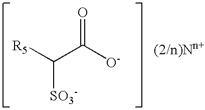
   wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
   wherein the ratio of i) to ii) is from about 10:1 to about 1:10;
(d) removing from about 50% to about 90% by weight of the total water from the second intermediate liquid mixture to form a thickened mixture; and
(e) extruding the thickened mixture to form flaked solid or semi-solid particles.

This process embodiment may further comprise plodding the flaked solid or semi-solid particles to form plodded particles, extruding the plodded particles to form a billet, cutting the billet, and stamping the cut billet to yield a personal cleansing or laundry detergent bar. In accordance with this embodiment, preferably R₁ is a C₁₆-C₁₈ hydrocarbyl group, an alkyl group, or combination thereof, and M is sodium or potassium, or a mixture thereof. Preferably, the soap is present from about 68% to about 78% by weight. Also Preferably, R₂ is a C₁₂-C₂₀ hydrocarbyl group, an alkyl group, or combination thereof. Preferred fatty acids include coconut fatty acids and stearic acid and coconut fatty acid mixtures. Further in accordance with this process embodiment, the fatty acid is preferably present from about 2% to about 7% by weight. Also in a preferred embodiment, R₃ is a C₈-C₂₀ hydrocarbyl group, an alkyl group, or combination thereof, R₄ is methyl and M is hydrogen, sodium, potassium, calcium, magnesium ammonium, monoethanolammonium, diethanolammonium, triethanolammonium, or a mixture thereof; R₅ is a C₈-C₂₀ hydrocarbyl group, an alkyl group, or combination thereof, and N is hydrogen, sodium, potassium, calcium, magnesium, ammonium, monoethanolammonium, diethanolammonium, triethanolammonium, or a mixture thereof. Ideally, the ratio of the mixture of anionic surfactants is from about 3:1 to about 1:3. Further the preferred salt is sodium chloride. Also more preferably, the polyhydridic alcohol is selected from the group consisting of glycerine, polyglycerol esters, sorbitol and propylene glycol, or a mixture thereof; most preferably the polyhydridic alcohol is glycerine. Also preferably, y is 2. In accordance with this process embodiment, removing the water from the liquid mixture is accomplished by scraped wall vacuum evaporation drying under reduced pressure or heated drum drying at ambient pressure. Preferably, about 55% to about 85% by weight of the water is removed from the liquid mixture; and most preferably, about 60% to about 80% by weight of the water is removed from the liquid mixture. The invention relates to a personal cleansing and laundry detergent bar pre-blend, produced by the process. Further in accordance with this embodiment, the invention relates to a personal cleansing and laundry detergent bar pre-blend, produced by the process and/or a personal cleansing and laundry bar produced by the process.

In another aspect, the invention relates to a process for preparing a personal cleansing and laundry detergent bar pre-blend, comprising the sequential steps of:
(a) forming at a temperature of about 65° C. to about 105° C. a substantially homogeneous aqueous soap-fatty acid liquid mixture comprising
   1. from about 58% to about 93% by weight of an approximately 70% aqueous soap slurry, the soap being of the formula 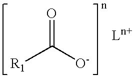
   wherein R₁ is a C₆-C₂₂ hydrocarbyl group, an alkyl group, or combination thereof, n is 1 or 2, and L is a cation; and
   2. from about 1% to about 15% by weight of a fatty acid of the formula 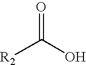
   wherein R₂ is a C₆-C₂₂ hydrocarbyl group, an alkyl group, or combination thereof; and
(b) forming at a temperature of about 65° C. to about 105° C. a liquid alcohol-salt-anionic surfactant mixture comprising
   1. from about 0.5% to about 2% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof; and
   2. from about 0.5% to about 10% by weight of a polyhydridic alcohol;
   3. from about 2% to about 30% by weight of an approximately 55% aqueous mixture of anionic surfactants, the anionic surfactants comprising
      i) an alpha sulfonated alkyl ester of the formula 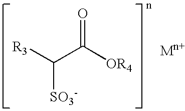
      wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
      ii) a sulfonated fatty acid of the formula 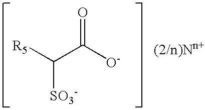
      wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
      wherein the ratio of i) to ii) is from about 10:1 to about 1:10;
(c) combining said liquid alcohol-salt-anionic surfactant mixture and said liquid soap-fatty acid mixture at a temperature of about 65° C. to about 105° C. to form an intermediate liquid mixture;
(d) optionally adding to said intermediate liquid mixture from 0 to about 10% by weight of an alkanolamide of the formula 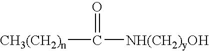
wherein n=6-16, and y is 2-4;
(e) removing from about 50% to about 90% by weight of the total water from the intermediate liquid mixture to form a thickened mixture; and
(f) extruding the thickened mixture to form flaked solid or semi-solid particles.

This process embodiment may further comprise plodding the flaked solid or semi-solid particles to form plodded particles, extruding the plodded particles to form a billet, cutting the billet, and stamping the cut billet to yield a personal cleansing or laundry detergent bar. In accordance with this embodiment, preferably R₁ is a C₆-C₁₈ hydrocarbyl group, an alkyl group, or combination thereof, and M is sodium or potassium, or a mixture thereof. Preferably, the soap is present from about 68% to about 78% by weight. Also preferably, R₂ is a C₁₂-C₂₀ hydrocarbyl group, an alkyl group, or combination thereof. Preferred fatty acids include coconut fatty acids and stearic acid and coconut fatty acid mixtures. Further in accordance with this process embodiment, the fatty acid is preferably present from about 2% to about 7% by weight. Also in a preferred embodiment, R₃ is a C₈-C₂₀ hydrocarbyl group, an alkyl group, or combination thereof, R₄ is methyl and M is hydrogen, sodium, potassium, calcium, magnesium ammonium, monoethanolammonium, diethanolammonium, triethanolammonium, or a mixture thereof; R₅ is a C₈-C₂₀ hydrocarbyl group, an alkyl group, or combination thereof, and N is hydrogen, sodium, potassium, calcium, magnesium, ammonium, monoethanolammonium, diethanolammonium, triethanolammonium, or a mixture thereof. Ideally, the ratio of the mixture of anionic surfactants is from about 3:1 to about 1:3. Further the preferred salt is sodium chloride. Also more preferably, the polyhydridic alcohol is selected from the group consisting of glycerine, polyglycerol esters, sorbitol and propylene glycol, or a mixture thereof; most preferably the polyhydridic alcohol is glycerine. Also preferably, y is 2. In accordance with this process embodiment, removing the water from the liquid mixture is accomplished by scraped wall vacuum evaporation drying under reduced pressure or heated drum drying at ambient pressure. Preferably, about 55% to about 85% by weight of the water is removed from the liquid mixture; and most preferably, about 60% to about 80% by weight of the water is removed from the liquid mixture. The invention relates to a personal cleansing and laundry detergent bar pre-blend, produced by the process. Further in accordance with this embodiment, the invention relates to a personal cleansing and laundry detergent bar pre-blend, produced by the process and/or a personal cleansing and laundry bar produced by the process.

In another aspect, the invention relates to a process for preparing a personal cleansing and laundry detergent bar pre-blend, comprising the sequential steps of:
(a) forming at a temperature of about 65° C. to about 105° C. a substantially homogeneous aqueous soap-fatty acid-anionic surfactant liquid mixture comprising
   1. from about 58% to about 93% by weight of an approximately 70% aqueous soap slurry, the soap being of the formula 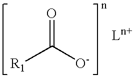
   wherein R₁ is a C₆-C₂₂ hydrocarbyl group, an alkyl group, or combination thereof, n is 1 or 2, and L is a cation; and
   2. from about 1% to about 15% by weight of a fatty acid of the formula 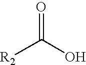
   wherein R₂ is a C₆-C₂₂ hydrocarbyl group, an alkyl group, or combination thereof; and
   3. from about 2% to about 15% by weight of an approximately 55% aqueous mixture of anionic surfactants, the anionic surfactants comprising
      i) an alpha sulfonated alkyl ester of the formula 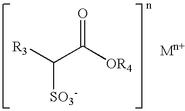
      wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
      ii) a sulfonated fatty acid of the formula 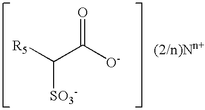
      wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
      wherein the ratio of i) to ii) is from about 10:1 to about 1:10;
(b) forming at a temperature of about 65° C. to about 105° C. a liquid alcohol-salt-anionic surfactant mixture comprising
   1. from about 0.5% to about 2% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof; and
   2. from about 0.5% to about 10% by weight of a polyhydridic alcohol;
   3. from about 3% to about 15% by weight of an approximately 55% aqueous mixture of anionic surfactants, the anionic surfactants comprising
      i) an alpha sulfonated alkyl ester of the formula 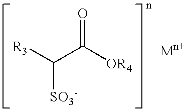
      wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
      ii) a sulfonated fatty acid of the formula 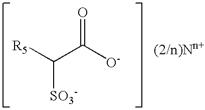
      wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
      wherein the ratio of i) to ii) is from about 10:1 to about 1:10;
(c) combining said liquid soap-fatty acid-anionic surfactant mixture and said liquid alcohol-salt-anionic surfactant mixture at a temperature of about 65° C. to about 105° C. to form an intermediate liquid mixture;
(d) optionally adding to said intermediate liquid mixture from 0 to about 10% by weight of an alkanolamide of the formula 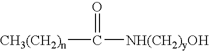
wherein n=6-16, and y is 2-4;
(e) removing from about 50% to about 90% by weight of the total water from the intermediate liquid mixture to form a thickened mixture; and
(f) extruding the thickened mixture to form flaked solid or semi-solid particles.
   This process embodiment may further comprise plodding the flaked solid or semi-solid particles to form plodded particles, extruding the plodded particles to form a billet, cutting the billet, and stamping the cut billet to yield a personal cleansing or laundry detergent bar. In accordance with this embodiment, preferably R₁ is a C₆-C₁₆ hydrocarbyl group, an alkyl group, or combination thereof, and M is sodium or potassium, or a mixture thereof. Preferably, the soap is present from about 68% to about 78% by weight. Also preferably, R₂ is a C₁₂-C₂₀ hydrocarbyl group, an alkyl group, or combination thereof. Preferred fatty acids include coconut fatty acids and stearic acid and coconut fatty acid mixtures. Further in accordance with this process embodiment, the fatty acid is preferably present from about 2% to about 7% by weight. Also in a preferred embodiment, R₃ is a C₈-C₂₀ hydrocarbyl group, an alkyl group, or combination thereof, R₄ is methyl and M is hydrogen, sodium, potassium, calcium, magnesium ammonium, monoethanolammonium, diethanolammonium, triethanolammonium, or a mixture thereof; R₅ is a C₈-C₂₀ hydrocarbyl group, an alkyl group, or combination thereof, and N is hydrogen, sodium, potassium, calcium, magnesium, ammonium, monoethanolammonium, diethanolammonium, triethanolammonium, or a mixture thereof. Ideally, the ratio of the mixture of anionic surfactants is from about 3:1 to about 1:3. Further the preferred salt is sodium chloride. Also more preferably, the polyhydridic alcohol is selected from the group consisting of glycerine, polyglycerol esters, sorbitol and propylene glycol, or a mixture thereof; most preferably the polyhydridic alcohol is glycerine. Also preferably, y is 2. In accordance with this process embodiment, removing the water from the liquid mixture is accomplished by scraped wall vacuum evaporation drying under reduced pressure or heated drum drying at ambient pressure. Preferably, about 55% to about 85% by weight of the water is removed from the liquid mixture; and most preferably, about 60% to about 80% by weight of the water is removed from the liquid mixture. The invention relates to a personal cleansing and laundry detergent bar pre-blend, produced by the process. Further in accordance with this embodiment, the invention relates to a personal cleansing and laundry detergent bar pre-blend, produced by the process and/or a personal cleansing and laundry bar produced by the process.

In yet another aspect, the invention relates to a composition suitable for formation into precursor cleansing/laundry bar soap noodles, personal cleansing bars and laundry detergent bars comprising:
(a) from about 58% to about 93% by weight of an approximately 70% aqueous soap slurry, the soap being of the formula 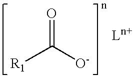
wherein R₁ is a C₆-C₂₂ hydrocarbyl group, an alkyl group, or combination thereof, n is 1 or 2, and L is a cation; and
(b) from about 1% to about 15% by weight of a fatty acid of the formula 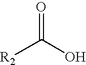
wherein R₂ is a C₆-C₂₂ hydrocarbyl group, an alkyl group, or combination thereof; and
(c) from about 2% to about 30% by weight of an approximately 55% aqueous mixture of anionic surfactants, the anionic surfactants comprising
   i) an alpha sulfonated alkyl ester of the formula 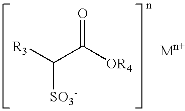
   wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
   ii) a sulfonated fatty acid of the formula 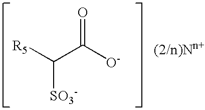
   wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
   wherein the ratio of i) to ii) is from about 10:1 to about 1:10;
(d) from about 0.5% to about 2% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof;
(e) from about 0.5% to about 5.0% by weight of a polyhydridic alcohol; and
(f) from 0 to about 10% by weight of an alkanolamide of the formula 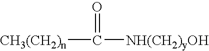
wherein n=6-16, and y is 2-4;
   This compositional embodiment may further comprise from about 1% to about 5% by weight paraffin. In accordance with this embodiment, preferably R₁ is a C₆-C₁₈ hydrocarbyl group, an alkyl group, or combination thereof, and M is sodium or potassium, or a mixture thereof. Preferably, the soap is present from about 68% to about 78% by weight. Also preferably, R₂ is a C₁₂-C₂₀ hydrocarbyl group, an alkyl group, or combination thereof. Preferred fatty acids include coconut fatty acids and stearic acid and coconut fatty acid mixtures. Further in accordance with this process embodiment, the fatty acid is preferably present from about 2% to about 7% by weight. Also preferably, y is 2. Also in a preferred embodiment, R₃ is a C₈-C₂₀ hydrocarbyl group, an alkyl group, or combination thereof, R₄ is methyl and M is hydrogen, sodium, potassium, calcium, magnesium ammonium, monoethanolammonium, diethanolammonium, triethanolammonium, or a mixture thereof; R₅ is a C₈-C₂₀ hydrocarbyl group, an alkyl group, or combination thereof, and N is hydrogen, sodium, potassium, calcium, magnesium, ammonium, monoethanolammonium, diethanolammonium, triethanolammonium, or a mixture thereof. Ideally, the ratio of the mixture of anionic surfactants is from about 3:1 to about 1:3. Further the preferred salt is sodium chloride. Also more preferably, the polyhydridic alcohol is selected from the group consisting of glycerine, polyglycerol esters, sorbitol and propylene glycol, or a mixture thereof; most preferably the polyhydridic alcohol is glycerine.

In yet another aspect, the invention relates to a personal cleansing/laundry detergent bar comprising:
(a) from about 50% to about 85% by weight of a soap of the formula 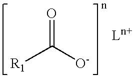
wherein R₁ is a C₆-C₂₂ hydrocarbyl group, an alkyl group, or combination thereof, n is 1 or 2, and L is a cation; and
(b) from about 1% to about 15% by weight of a fatty acid of the formula 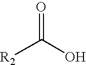
wherein R₂ is a C₆-C₂₂ hydrocarbyl group, an alkyl group, or combination thereof; and
(c) from about 3.5% to about 20% by weight of a mixture of anionic surfactants comprising
   i) an alpha sulfonated alkyl ester of the formula 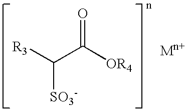
   wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
   ii) a sulfonated fatty acid of the formula 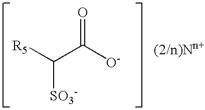
   wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
   wherein the ratio of i) to ii) is from about 10:1 to about 1:10;
(d) from about 0.7% to about 3% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof;
(e) from about 0.5% to about 6% by weight of a polyhydridic alcohol;
(f) from 0 to about 10% by weight of an alkanolamide of the formula 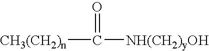
wherein n=6-16, and y is 2-4; and
(g) from about 3% to about 16% by weight of water.
   This bar composition may further comprise from about 1% to about 5% by weight paraffin. In accordance with this embodiment, preferably R₁ is a C₆-C₁₈ hydrocarbyl group, an alkyl group, or combination thereof, and M is sodium or potassium, or a mixture thereof. Preferably, the soap is present from about 68% to about 78% by weight. Also preferably, R₂ is a C₁₂-C₂₀ hydrocarbyl group, an alkyl group, or combination thereof. Preferred fatty acids include coconut fatty acids and stearic acid and coconut fatty acid mixtures. Further in accordance with this process embodiment, the fatty acid is preferably present from about 2 to about 7% by weight. Also in a preferred embodiment, R₃ is a CB-C₂₀ hydrocarbyl group, an alkyl group, or combination thereof, R₄ is methyl and M is hydrogen, sodium, potassium, calcium, magnesium ammonium, monoethanolammonium, diethanolammonium, triethanolammonium, or a mixture thereof; R₅ is a C₈-C₂₀ hydrocarbyl group, an alkyl group, or combination thereof, and N is hydrogen, sodium, potassium, calcium, magnesium, ammonium, monoethanolammonium, diethanolammonium, triethanolammonium, or a mixture thereof. Ideally, the ratio of the mixture of anionic surfactants is from about 3:1 to about 1:3. Further the preferred salt is sodium chloride. Also more preferably, the polyhydridic alcohol is selected from the group consisting of glycerine, polyglycerol esters, sorbitol and propylene glycol, or a mixture thereof; most preferably the polyhydridic alcohol is glycerine. Further preferably, y is 2.

As previously stated, compositions and the methods of producing such compositions of the invention contain (or utilize) about 0.5% to about 2% by weight of a salt. Generally, without being bound by any particular theory, the salt may be any such salt capable of acting as crisping agent or builder to a final bar formulation. Preferably, salt is selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or mixtures thereof. In a more preferred embodiment of the present invention the salt is magnesium chloride, sodium chloride or a mixture thereof. In a most preferred embodiment the salt is sodium chloride.

The compositions and the methods of producing such compositions also optionally may further comprise (or utilize) additional ingredients including from about 0.5% to about 10% by weight of a sucrogylceride, a functional metallic soap, a succinamate, a sulfosuccinamate, a mono-, di-, or trigylceride, chitosan, or a mixture thereof. Similarly, the compositions and the methods of producing such compositions may further comprise (or utilize) from about 0.1% to about 10% by weight of fragrance, emollients, moisturizers, viscosity control agents, as well as additional agents appropriate for incorporation into a composition of the invention and which are known to those skilled in the art.

The compositions of the invention may be transparent and/or produce a transparent personal cleansing or laundry detergent bar upon proper processing and/or selection of optional ingredients and components detailed herein. Additionally, the compositions may be used to produce a transparent dish washing gel, paste or solution, or further applications such as are apparent to one skilled in the art. Whether transparent or nontransparent, the compositions may exist as solid flakes, or as a gel.

All numerical limits, ranges, ratios, etc., are approximations (“abouts”) unless otherwise specified. Within the scope of the invention, there are several different preferred embodiments.

The term “soap” as used herein includes the plural as well as the singular in terms of mixed ions and fatty acid chains unless otherwise specified.

The terms “coconut oil” (CNO); “palm kernel oil” (PKO); “palm oil stearin” (POS); and “tallow” (T) as used herein refer to a mixture of soaps having an approximate chain length distribution as usually defined in the literature; unless otherwise specified.

Alpha Sulfonated Alkyl Esters and Alpha Sulfonated Fatty Acids

The compositions of the invention and the methods of producing such compositions typically contain (or utilize) from about 2% to about 30% by weight of an approximately 55% aqueous mixture of an anionic surfactants comprising an alpha sulfonated alkyl ester and a sulfonated fatty acid. The alpha sulfonated alkyl esters used in the invention are typically prepared by sulfonating an alkyl ester of a fatty acid with a sulfonating agent such as SO₃, followed by neutralization with a base, such as sodium hydroxide, potassium hydroxide, calcium hydroxide, magnesium oxide, monoethanolamine, diethanolamine or triethanolamine, or a mixture thereof. When prepared in this manner, the alpha sulfonated alkyl esters normally contain a minor amount, typically not exceeding 33% by weight, of alpha sulfonated fatty acid, i.e., disalt, which results from hydrolysis of the ester. Generally, larger amounts of the disalt are obtained by hydrolyzing a known amount of the monosalt; hydrolysis may be accomplished in situ during the preparation of the composition. Accordingly, the alpha sulfonated alkyl ester and alpha sulfonated fatty acid may be provided to the composition or utilized in the inventive process as a blend of components which naturally result from the sulfonation of an alkyl ester of a fatty acid, or as individual components. Furthermore, it is known to one skilled in the art that minor impurities such as sodium sulfate, unsulfonated methyl esters (ME), and unsulfonated fatty acids (FA) may also be present in the mixtures according to the invention.

The alpha sulfonated alkyl esters, i.e., alkyl ester sulfonate surfactants, include linear esters of C₆-C₂₂ carboxylic acid (i.e., fatty acids) which are sulfonated with gaseous SO₃ according to the “The Journal of American Oil Chemists Society,” 52 (1975), pp. 323-329. Suitable starting materials include, among others, natural fatty substances as derived from tallow, palm oil, etc.

In some embodiments of the invention the α-sulfonated alkyl ester is a sulfonated methyl ester, desirably as further described herein. Accordingly, the invention, in some embodiments, provides a composition and the methods of producing such compositions wherein the alpha sulfonated alkyl ester is of the formula 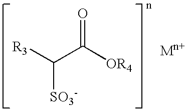
wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof.

The invention further provides a composition and the methods of producing such composition wherein the sulfonated fatty acid is of the formula 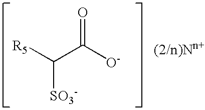
wherein in some embodiments R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof.

Fatty Acids

The compositions and the methods of producing such compositions of the invention typically contain (or utilize) from about 1% to about 15% by weight of a fatty acid. The (free) fatty acids used in the invention correspond with the fatty acids used to make conventional soaps. The fatty acid material which is desirably incorporated into the invention includes material ranging in hydrocarbon chain length of from about 6 to about 22, essentially saturated. These fatty acids can be highly purified individual chain lengths and/or crude mixtures such as those derived from fats and oils. The industry term “triple pressed stearic acid” comprises about 45 parts stearic and 55 parts palmitic acids. Additionally, the term stearic acid is used in the context of the soap industry to refer to a fatty acid mixture which is predominately stearic acid. Thus, this is its meaning as used herein.

The composition and the methods of producing such compositions may include soaps derived from hydrocarbon chain lengths of from about 6 to about 22 (including carboxyl carbon) and, in some embodiments of the invention, are saturated. In some manifestations of this embodiment, the soap is the sodium salt, but other soluble soap can be used. Potassium, calcium, magnesium, monoethanolammonium; diethanolammonium, triethanolammonium, and mixtures thereof, are deemed acceptable. Thus the counterion, L, aqueous soap slurry in the above description is a cation that is preferably selected from sodium, potassium, calcium, magnesium, ammonium, monoethanolammonium, diethanolammonium, triethanolammonium, and a mixture thereof. The soaps can be prepared by the in situ saponification or ion exchange with halide salt of the corresponding fatty acids, but they may also be introduced as preformed soaps.

Polyhydridic Alcohols

The polyhydridic alcohol may be a polyol generally defined as a non-volatile di- or higher polyhydridic alcohol, a sugar or a polyethylene glycol. Particular examples include glycerine, propylene glycol, glycerol, sorbitol, sucrose and 200-400 molecular weight polyethylene glycol, dipropylene glycol, polypropylene glycols 2000, 4000, polyoxyethylene polyoxypropylene glycols, polyoxypropylene polyoxyethylene glycols, glycerol, sorbitol, ethoxylated sorbitol, hydroxypropyl sorbitol, polyethylene glycol 200-6000, methoxy polyethylene glycols 350, 550, 750, 2000, 5000, poly[ethylene oxide] homopolymers (100,000-5,000,000), polyalkylene glycols and derivatives, hexylene glycol (2-methyl-2,4-pentanediol), 1,3-butylene glycol, 1,2,6-hexanetriol, ethohexadiol USP (2-ethyl-1,3-hexanediol), C₁₅-C₁₈ vicinal glycol, and polyoxypropylene derivatives of trimethylolpropane are examples of this class of materials.

The useful polyols of the invention are liquid water-soluble aliphatic polyols or polyethylene glycols or polypropylene glycols. The polyol may be saturated or contain ethylenic linkages; it must have at least two alcohol groups attached to separate carbon atoms in the chain, and must be water soluble and liquid at room temperature. If desired, the compound may have an alcohol group attached to each carbon atom in the chain. Among the compounds which are effective are ethylene glycol, propylene glycol, glycerine and mixtures thereof. In some embodiments of the invention the polyol is glycerine. Water-soluble polyethylene glycols, water-soluble polypropylene glycols useful in the present invention are those products produced by the condensation of ethylene glycol molecules or propylene glycol molecules to form high molecular weight ethers having terminal hydroxyl groups. The polyethylene glycol compounds may range from diethylene glycol to those having molecular weights as high as about 800, and, in some embodiments, about 100 to 700, in other embodiments, 100 to 600. Normally, polyethylene glycols having molecular weights up to 800 are liquid and completely soluble in water. As the molecular weight of the polyethylene glycol increases beyond 800, they become solid and less water-soluble. Such solids may be used as plasticizers herein when malleable at 35° C. to about 46° C. The polypropylene glycol compounds useful in this invention may range from dipropylene glycol to polypropylene glycols having molecular weights of about 2000, and, in some embodiments, less than 1500, in other embodiments, less than 1000. These are normally liquid at room temperature and are readily soluble in water.

Composition pH

Although not critical, the compositions and the methods of producing such compositions herein may be formulated and carried out such that they will have a pH of between about 4.0 and about 10.0, and, in some embodiments, between about 5 and about 9.5. Techniques for controlling pH at recommended usage levels include the use of buffers, alkali, acids, etc., and are well known to those skilled in the art.

### Optional Components

Synthetic Detergent Surfactants

The invention encompasses the optional use of additional synthetic detergent surfactants, such as for example, acyl isethionates, e.g., sodium acyl (cocoyl) isethionate (SCI). In some embodiments of the invention, the SCI is “STCI” herein defined as “sodium topped coconut isethionate” which is further defined as SCI with alkyl carbon chains having: 0% to 4% of highly soluble acyl groups (C₆, C₈, C₁₀, C_{18:1}, and C_{18:2}), 45-65% C₁₂, and 30%-55% C₁₄, C₁₆, C₁₈. The terms SCI and STCI are used interchangeably herein unless otherwise specified.

Additional optional detergent surfactants include, among others, anionic, zwitterionic, amphoteric, semi-polar nonionic, or nonionic, or mixtures thereof.

Examples of useful optional anionic surfactants include, among others, the sodium, potassium, magnesium, calcium, ammonium, monoethanolammonium (MEA), diethanolammonium (DEA), triethanolammonium (TEA), or alkyl amine salts, or mixtures thereof, of sulfonic acids, polysulfonic acids, sulfonic acids of oils, paraffin sulfonic acids, lignin sulfonic acids, petroleum sulfonic acids, tall oil acids, olefin sulfonic acids, hydroxyolefin sulfonic acids, polyolefin sulfonic acids, polyhydroxy polyolefin sulfonic acids, perfluorinated carboxylic acids, alkoxylated carboxylic acid sulfonic acids, polycarboxylic acids, polycarboxylic acid polysulfonic acids, alkoxylated polycarboxylic acid polysulfonic acids, phosphoric acids, alkoxylated phosphoric acids, polyphosphoric acids, and alkoxylated polyphosphoric acids, fluorinated phosphoric acids, phosphoric acid esters of oils, phosphinic acids, alkylphosphinic acids, aminophosphinic acids, polyphosphinic acids, vinyl phosphinic acids, phosphonic acids, polyphosphonic acids, phosphonic acid alkyl esters, α-phosphono fatty acids, oragnoamine polymethylphosphonic acids, organoamino dialkylene phosphonic acids, alkanolamine phosphonic acids, trialkyledine phosphonic acids, acylamidomethane phosphonic acids, alkyliminodimethylene diphosphonic acids, polymethylene-bis(nitrilo dimethylene)tetraphosphonic acids, alkyl bis(phosphonoalkylidene) amine oxide acids, esters of substituted aminomethylphosphonic acids, phosphonamidic acids, acylated amino acids (e.g., amino acids reacted with alkyl acyl chlorides, alkyl esters or carboxylic acids to produce N-acylamino acids), N-alkyl acylamino acids, acylated protein hydrolysates, branched alkylbenzene sulfonic acids, alkyl gylceryl ether sulfuric acid esters, alkyl sulfuric acid esters, alkoxylated alkyl sulfuric acid esters, α-sulfonated ester diacids, alkoxylated α-sulfonated alkyl ester acids, α-sulfonated dialkyl diester acids, di-α-sulfonated dialkyl diester acids, α-sulfonated alkyl acetate acids, primary and secondary alkyl sulfonic acids, perfluorinated alkyl sulfonic acids, sulfosuccinic mono- and diester acids, polysulfosuccinic polyester acids, sulfoitaconic diester acids, sulfosuccinamic acids, sulfosuccinic amide acids, sulfosuccinic imide acids, phthalic acids, sulfophthalic acids, sulfoisophthalic acids, phthalamic acids, sulfophthalamic acids, alkyl ketone sulfonic acids, hydroxyalkane-1-sulfonic acids, lactone sulfonic acids, sulfonic acid amides, sulfonic acid diamides, alkyl phenol sulfuric acid esters, alkoxylated alkyl phenol sulfuric acid esters, alkylated cycloalkyl sulfuric acid esters, alkoxylated alkylated cycloalkyl sulfuric acid esters, dendritic polysulfonic acids, dendritic polycarboxylic acids, dendritic polyphosphoric acids, sarcosinic acids, isethionic acids, tauric acids, fluorinated carboxylic acids, fluorinated sulfonic acids, fluorinated sulfate acids, fluorinated phosphonic and phosphinic acids, and mixtures thereof.

Suitable optional nonionic surfactants in accordance with the invention are disclosed in U.S. Pat. No. 3,929,678, Laughlin et al., issued Dec. 30, 1975, at column, 13 line 14 through column 16, line 6, incorporated herein by reference. Generally, the nonionic surfactant is selected from the group comprising polyoxyethyleneated alkylphenols, polyoxyethyleneated straight chain alcohols, polyoxyethyleneated branched chain alcohols, polyoxyethyleneated polyoxypropylene glycols, polyoxyethyleneated mercaptans, fatty acid esters, glyceryl fatty acid esters, polyglyceryl fatty acid esters, propylene glycol esters, sorbitol esters, polyoxyethyleneated sorbitol esters, polyoxyethylene glycol esters, polyoxyethyleneated fatty acid esters, primary alkanolamides, ethoxylated primary alkanolamides, secondary alkanolamides, ethoxylated secondary alkanolamides, tertiary acetylenic glycols, polyoxyethyleneated silicones, N-alkylpyrrolidones, alkylpolyglycosides, alkylpolylsaccharides, EO-PO block polymers, polyhydroxy fatty acid amides, amine oxides and mixtures thereof. Further, exemplary, non-limiting classes of useful nonionic surfactants are listed below:

1. The polyethylene, polypropylene, and polybutylene oxide condensates of alkyl phenols. These compounds include the condensation products of alkyl phenols having an alkyl group containing from about 6 to 12 carbon atoms in either a straight or branched chain configuration with the alkylene oxide. In some embodiments, the polyethylene oxide condensates are used and is present in an amount equal to from about 1 to about 25 moles of ethylene oxide per mole of alkyl phenol. Commercially available nonionic surfactants of this type include Igepal® CO-630, marketed by the GAF Corporation; and Triton® X-45, X-114, X-100 and X-102, all marketed by the Rohm and Haas Company.

2. The condensation products of aliphatic alcohols with from about 1 to about 25 moles of ethylene oxide. The alkyl chain of the aliphatic alcohol can either be straight or branched, primary or secondary, and contain from about 8 to about 22 carbon atoms. In some embodiments of the invention the condensation products of alcohols having an alkyl group containing from about 6 to about 11 carbon atoms with from about 2 to about 10 moles of ethylene oxide per mole of alcohol are used. Examples of commercially available nonionic surfactants of this type include Tergitol® 15-S-9 (the condensation products of C₁₁-C₁₅ linear alcohol with 9 moles of ethylene oxide), Tergitol® 24-L-6 NMW (the condensation products of C₁₂-C₁₄ primary alcohol with 6 moles of ethylene oxide with a narrow molecular weight distribution), both marketed by Union Carbide Corporation; Neodol® 91-8 (the condensation product of C₉-C₁₁ linear alcohol with 8 moles of ethylene oxide), Neodol® 23-6.5 (the condensation product of C₁₂-C₁₃ linear alcohol with 6.5 moles of ethylene oxide), Neodol® 45-7 (the condensation product of C₁₄-C₁₅ linear alcohol with 7 moles of ethylene oxide), Neodol® 91-6 (the condensation product of C₉-C₁₁ linear alcohol with 6 moles of ethylene oxide), marketed by Shell Chemical Company, and Kyro® EOB (the condensation product of C₁₃-C₁₅ linear alcohol with 9 moles of ethylene oxide), marketed by the Procter and Gamble Company.

3. The condensation products of ethylene oxide with a hydrophobic base formed by the condensation of propylene oxide with propylene glycol. The hydrophobic portion of these compounds, in some embodiments, has a molecular weight of from about 1500 to about 1880 and exhibits water insolubility. The addition of polyoxyethylene moieties to this hydrophobic portion tends to increase the water solubility of the molecule as a whole, and the liquid character of the product is retained up to the point where the polyoxyethylene content is about 50% of the total weight of the condensation product, which corresponds to condensation with up to about 40 moles of ethylene oxide. Examples of compounds of this type include certain of the commercially available Pluronic® surfactants, marketed by BASF.

4. The condensation products of ethylene oxide with the product resulting from the reaction of propylene oxide and ethylenediamine. The hydrophobic moiety of these products consists of the reaction product of ethylenediamine and excess propylene oxide, and has a molecular weight of from about 2500 to about 3000. This hydrophobic moiety is condensed with ethylene oxide to the extent that the condensation product contains from about 40% to about 80% by weight of polyoxyethylene and has a molecular weight of from about 5,000 to about 11,000. Examples of this type of nonionic surfactant include certain of the commercially available Tetronic® compounds, marketed by BASF.

5. Semi-polar nonionic surfactants are a special category of nonionic surfactants which include water-soluble amine oxides containing on alkyl moiety of from about 10 to about 18 carbon atoms and 2 moieties selected from the group comprising alkyl groups and hydroxyalkyl groups containing from about 1 to about 3 carbon atoms; and water-soluble sulfoxides containing alkyl moieties of from about 10 to about 18 carbon atoms and a moiety selected from the group comprising alkyl groups and hydroxyalkyl groups of from about 1 to about 3 carbon atoms.

6. Alkylpolysaccharides disclosed in U.S. Pat. No. 4,565,647, Lenado, issued Jan. 21, 1986, incorporated herein by reference, having a hydrophobic group containing from about 6 to about 30 carbon atoms, in some embodiments from about 10 to about 16 carbon atoms, and a polysaccharide, e.g., a polyglucoside, hydrophilic group containing from about 1.3 to about 10, in some embodiments from about 1.3 to about 3, and in other embodiments about 1.3 to about 2.7 saccharide units. Any reducing saccharide containing 5 or 6 carbon atoms can be used, e.g., glucose, galactose and galactosyl moieties can be substituted for the glucosyl moieties. (Optionally, the hydrophobic group is attached at the 2-, 3-, 4-, etc. positions thus giving a glucose or galactose as opposed to a glucoside or galactoside.) The intersaccharide bonds can be, e.g., between the one position of the additional saccharide units and the 2-, 3-, 4-, and/or 6-positions on the preceding saccharide units.

7. An ethyl ester ethoxylate and/or alkoxylate such as those described in U.S. Pat. No. 5,220,046, incorporated herein by reference. These material may be prepared according to the procedure set forth in Japanese Kokai patent application No. HEI 5 [1993]-22396. For example, they may be prepared by a one-step condensation reaction between an alkyl ester and an alkylene oxide in the presence of a catalytic amount of magnesium together with another ion selected from the group of Al⁺³, Ga⁺³, In⁺³, Co⁺³, Sc⁺³, La⁺³ and Mn⁺³. Optionally, and less desirably, there can be a polyalkyleneoxide chain joining the hydrophobic moiety and the polysaccharide moiety. In some embodiments of the invention, the alkyleneoxide is ethylene oxide. Typical hydrophobic groups include alkyl groups, either saturated or unsaturated, branched or unbranched, containing from about 8 to about 18, in some embodiments from about 12 to about 14 carbon atoms; n is 2 or 3, and in some embodiments it is 2; t is from about 0 to about 10, and in some embodiments it is 0; and x is from about 1.3 to about 10, in some embodiments it is from about 1.3 to 3, in other embodiments it is from about 1.3 to about 2.7. The glycosyl can be derived from glucose. To prepare these compounds, the alcohol or alkylpolyethoxy alcohol is formed first and then reacted with glucose, or a source of glucose, to form the glucoside (attachment at the 1-position). The additional glucosyl units can then be attached between their 1-position and the preceding glycosyl units 2-, 3-, 4-, and/or 6-position, and in some embodiments predominately the 2-position.

Suitable optional amphoteric surfactants are selected from the group comprising alkyl glycinates, propionates, imidazolines, amphoalkylsulfonates sold as “Miranol”® by Rhone Poulenc, N-alkylaminopropionic acids, N-alkyliminodipropionic acids, imidazoline carboxylates, N-alkylbetaines, amido propyl betaines, sarcosinates, cocoamphocarboxyglycinates, amine oxides, sulfobetaines, sultaines and mixtures thereof. Additional suitable amphoteric surfactants include cocoamphoglycinate, cocoamphocarboxyglycinate, lauramphocarboxyglycinate, coco-amphopropionate, lauramphopropionate, stearamphoglycinate, cocoamphocarboxypropionate, tallowamphopropionate, tallowamphoglycinate, oleoamphoglycinate, caproamphoglycinate, caprylamphopropionate, caprylamphocarboxyglycinate, cocoyl imidazoline, lauryl imidazoline, stearyl imidazoline, behenyl imidazoline, behenylhydroxyethyl imidazoline, capryamphopropylsulfonate, cocamphopropylsulfonate, stearamphopropylsulfonate, oleoampho-propylsulfonate and the like.

Optional amine oxide surfactants which are suitable for use in the invention are alkylamine and amidoamine oxides. Examples of betaines and sultaines which are suitable for use in the invention are alkyl betaines and sultaines sold as “Mirataine”® by Rhone Poulenc, “Lonzaine”® by Lonza, Inc., Fairlawn, N.J. Examples of betaines and sultaines are cocobetaine, cocoamidoethyl betaine, cocoamidopropyl betaine, lauryl betaine, lauramidopropyl betaine, palmamidopropyl betaine, stearamidopropyl betaine, stearyl betaine, cocosultaine, lauryl sultaine, tallowamidopropyl hydroxysultaine and the like.

Optional pH adjusting agents are selected from the group comprising citric acid, succinic acid, phosphoric acid, sodium hydroxide, sodium carbonate, etc.

Optional sequestering agents are selected from the group comprising disodium ethylenediamine tetraacetate.

Additional optional auxiliary surfactants are selected from the group comprising amides, amine oxides, betaines, sultaines and C₈-C₁₈ fatty alcohols.

Examples of optional amine oxides in the invention include long-chain amine oxides, i.e., those compounds having the general formula 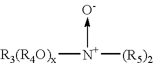
wherein R₃ is selected from an alkyl, hydroxyalkyl, acylamidopropyl and alkyl phenyl group, or mixtures thereof, containing from about 8-26 carbon atoms, in some embodiments from about 8-16 carbon atoms; R₄ is an alkylene or hydroxyalkylene group containing from about 2-3 carbon atoms, in some embodiments 2 carbon atoms, or mixtures thereof; x is from about 0-3, in some embodiments 0; and each R₅ is an alkyl or hydroxyalkyl group containing from about 1-3, in some embodiments from about 1-2 carbon atoms, or a polyethylene oxide group containing from about 1-3, in some embodiments 1, ethylene oxide groups. The R₅ groups can-be attached to each other, e.g., through an oxygen or nitrogen atom, to form a ring structure.

In some embodiments of the invention, the optional amine oxide surfactants include C₁₀-C₁₈ alkyl dimethyl amine oxides and C₈-C₁₂ alkoxy ethyl dihydroxyethyl amine oxides. Examples of such materials include dimethyloctylamine oxide, diethyldodecylamine oxide, bis-(2-hydroxyethyl)dodecylamine oxide, dimethyldodecylamine oxide, dodecylamidopropyl dimethylamine oxide and dimethyl-2-hydroxyoctadecylamine oxide. In some embodiments, C₁₀-C₁₈ alkyl dimethylamine oxide, and C₁₀-C₁₈ acylamido alkyl dimethylamine oxide are used.

Optional betaines useful surfactants in the invention include compounds having the formula R(R₁)₂N⁺R₂COO⁻ wherein R is a C₆-C₁₈ hydrocarbyl group, in some embodiments C₁₀-C₁₆ alkyl group, each R₁ is typically C₁-C₃, alkyl, in some embodiments methyl, and R₂ is a C₁-C₅ hydrocarbyl group, in some embodiments a C₁-C₅ alkylene group, in other embodiments a C₁-C₂ alkylene group. Examples of suitable betaines include coconut acylamidopropyldimethyl betaine; hexadecyl dimethyl betaine; C₁₂-C₁₄ acylamidopropylbetaine; C₈-C₁₄ acylamidohexyldiethyl betaine; 4-[C₁₄-C₁₆ acylmethylamidodiethylammonio]-1-carboxybutane; C₁₆-C₁₈ acylamidododimethylbataine; C₁₂-C₁₆ acylamidopentanediethylbetaine; C₁₂-C₁₆ acylmethylamidodimethylbetaine. In some embodiments the betaines are C₁₂-C₁₈ dimethylamoniohexanoate and the C₁₀-C₁₈ acylamidopropane (or ethane) dimethyl (or diethyl) betaines.

Optional sultaines useful surfactants in the invention include compounds having the formula R(R₁)₂N⁺R₂SO₃⁻, wherein R is a C₆-C₁₈ hydrocarbyl group, in some embodiments a C₁₀-C₁₆ alkyl group, in other embodiments a C₁₂-C₁₃ alkyl group; each R₁ is typically C₁-C₃ alkyl, in some embodiments methyl and R₂ is a C₁-C₆ hydrocabyl group, in some embodiments a C₁-C₃ alkylene or, in some embodiments, hydroxyalkylene group. Examples of suitable sultaines are C₁₂-C₁₄ dihydroxyethylammino propane sulfonate, and C₁₆-C₁₈ dimethylammonio hexane sulfonate, with C₁₂-C₁₄ amido propyl ammonio-2-hydroxypropyl sultaine being used in some embodiments.

Fatty acid amide surfactants are also optional components of the invention. In some embodiments amides are C₈-C₂₀ alkanol amides, monoethanolamides, diethanolamides and isopropanolamides. In another embodiment, the amide is a mixture of myristic monoethanolamide and lauric monoethanolamide. This amide is sold by Stepan Company, Northfield, Ill. as Ninol LMP. Other alkanolamides which optionally be included in the formulations of this invention are NINOL® COMF (available from Stepan Company) and NINOL® CMP (available from Stepan Company).

Other optional ingredients for use in the present compositions include non-volatile, nonionic silicone conditioning agents, polyalkyl or polyaryl siloxanes, and pearlescent/suspending agents, detergent builders, anti-bacterial agents, fluorescers, dyes or pigments, polymers, perfumes, cellulase enzymes, softening clays, smectite-type softening clays, polymeric clays, flocculating agents, dye transfer inhibitors, and optical brighteners.

Paraffins and Waxes

The compositions of the invention and the methods of producing such compositions may optionally contain (or utilize) about 1.0% to about 15.0% by weight of wax, in some embodiments paraffin, having a melting point of from about 54° C. to about 180° C. The waxes are selected from the group consisting of beeswax, spermaceti, carnauba, bayberry, candelilla, montan, ozokerite, ceresin, paraffin, synthetic waxes such as Fisher-Tropsch waxes, microcrystalline wax, and mixtures thereof. The wax ingredient is used in the product to impart skin mildness, plasticity, firmness, and processability. It also provides a glossy look and smooth feel to the bar.

A component of this invention can be a wax, and in some embodiments, paraffin wax having a melting point of from about 54° C. to about 82° C., in other embodiments from about 60° C. to about 74° C., and in yet other embodiments from about 61° C. to about 71° C. “High melt” paraffin is paraffin that has a melting point from about 66° C. to about 71° C. “Low melt” paraffin is paraffin that has a melting point from about 54° C. to about 60° C. In some embodiments, the paraffin wax is a fully refined petroleum wax which is odorless and tasteless and meets FDA requirements for use as coatings for food and food packages. Such paraffins are readily available commercially. A very suitable paraffin can be obtained, for example, from The National Wax Co. under the trade name 6975.

Cationic Polymers

The compositions and the methods of producing such compositions of the invention can optionally contain (or utilize) from about 0.5% to about 2% by weight of a suitably fast hydrating cationic polymer. The polymers have molecular weights of from about 1,000 to about 5,000,000. The cationic polymer (skin conditioning agent) is selected, e.g., from the group consisting of: (I) cationic polysaccharides; (II) cationic copolymers of saccharides and synthetic cationic monomers, and (III) synthetic polymers selected from the group consisting of: (A) cationic polyalkylene imines; (B) cationic ethoxy polyalkylene imines; and (C) cationic poly[N-[(-3-(dimethylammonio)propyl]-N′-[3-(ethyleneoxyethylene dimethylammonio)propyl]urea dichloride].

Plasticizers

The compositions of the invention and the methods of producing such compositions can optionally contain (or utilize) from about 1.0% to about 5.0% by weight of plasticizers. The plasticizers may be comprised of solid aliphatic materials. E.g. fatty alcohols, paraffins, monoglycerides, diglycerides, triglycerides, alkali soaps, alkaline soaps, or high molecular weight (solid) hydrophilic materials, e.g. polyethylene glycols, polypropylene glycols, starches, sugars and/or mixtures thereof.

Other Optional Ingredients

Other ingredients of the invention are selected for the various applications. E.g., perfumes can be used in formulating the skin cleansing products, at a level of from about 0.1 parts to about 1.5 parts of the composition. Vegetable oils, such as peanut and soybean oil, can be added at levels up to 10 parts, in some embodiments 2-6 parts. Alcohols, hydrotropes, colorants, and fillers such as talc, clay, calcium carbonate, oils and dextrin can also be used at appropriate levels. Preservatives, e.g., trisodium etidronate and sodium ethylenediaminetetraacetate (EDTA)., at a level of less than 1 parts of the composition, can be incorporated in the cleansing products to prevent color and odor degradation. Antibacterials can also be incorporated, usually at levels up to 1.5 parts. Salts, both organic and inorganic, can be incorporated. Examples include sodium chloride, sodium isethionate, sodium sulfate, and their equivalents.

Optional Adjunct Odor-Reducing or Odor-Controlling Materials

The compositions and the methods of producing such compositions of this invention can also contain (or utilize) an effective, i.e., odor-controlling, amount of various additional aluminosilicate and non-aluminosilicate odor-controlling materials to further expand their capacity for controlling odors, as well as the range of odor types being controlled. Such materials include, for example, cetyl pyridinium chloride, zinc chloride, EDTA, etidronate, BHT, and the like.

In some embodiments of the invention an aluminosilicate used is substantially free of particles sized greater than 30 microns, and in fact is substantially free of particles sized over 15 microns for acceptable bar feel. “Substantially free” means that the larger particles are less than about 5 parts, in some embodiments less than about 4 parts, in other embodiments less than about 3 parts, as measured by laser light scattering.

Optional Skin-Feel Enhancement Materials

The compositions and the methods of producing such compositions of this invention may contain (or utilize) an effective, i.e., skin softening and/or moisturizing, amount of various skin feel agents. These skin feel agents include, for example, chitan, triglycerides, glycerine, succinamates, sucroglycerides, and functional metallo-soaps. Suitable sucroglycerides are described in Pat. App. No. 96933018.2 (PCT/US96/14740) incorporated herein by reference. Suitable functional metallo-soaps are described in U.S. Pat. No. 4,921,942 (to Stepan Company), incorporated herein by reference.

While compositions of the invention are extremely useful in soap bar and laundry bar applications, other applications for these compositions are possible. The compositions of the invention may be useable in or as liquid, paste or gel dish washing compositions, hand soaps including waterless hand cleaners, multi-purpose cleaners, body washes, further laundry detergent compositions such as laundry powder, pre-spotter or stain sticks, textile treatment compositions including triethanolamine (TEA) soaps for dry cleaning, shampoos including those for humans, pets, and carpets, car wash, soap scouring pads and scrubbing pads, toilet tank drop ins and/or cleaners, personal care creams and lotions, and the like.

The definitions, abbreviations, and CTFA designations used in the invention are as set forth in Table 1

TABLE 1
Definitions, Abbreviations, and CTFA Designations

| | |
|---|---|
| BHT | butylated hydroxytoluene (di-tert-butyl-p- |
| | cresol) |
| BHA | butylated hydroxyanisole (3-t-butyl-4- |
| | hydroxyanisole) |
| Coco Fatty Acid | Emery 627 (a tradename from Emery |
| | Corporation, a division of Henkel) and |
| | coconut fatty acids that can be |
| | substituted for Emery 627 |
| EDTA | ethylenediamine tetraacetic acid |
| Hyamine | di-isobutyl-phenoxy-ethoxy-ethyl-dimethyl- |
| | benzyl ammonium chloride |
| MC-48 | average 6:1 mixture (i.e., ranging from |
| | 5:1 to 7:1) of sulfonated stripped coconut |
| | methyl esters and coconut fatty acids |
| Pristerene 4981 | Stearic Acid (from Unichema); approx. |
| | iodine value of 1.0 max.; mixture of about |
| | 65% C₁₈ fatty acid, about 28% C₁₆ fatty |
| | acid and about 2% myristic fatty acid |
| SFA | disalt; α-sulfonated fatty acid (e.g., |
| | that results from hydrolysis of SME) |
| SME | monosalt; α-sulfonated alkyl ester (e.g., |
| | α-sulfonated methyl ester) |
| UA | unreacted methyl ester |
| Alpha Step ® BSS-45 | An alpha sulfonated methyl ester available |
| | from Stepan Company, with the following |
| | properties: Average chain Length = 13.6; |
| | Sodium SME/SFA Actives = 43-45%; SME/SFA |
| | Ratio = 1.3-1.8:1; Solids = 53-55%; |
| | Inorganic Salts = 5-7%; Water = 45-48%; |
| | Free Oil = 1-3%; Working pH = 4-9. |

The invention is illustrated in the following non-limiting Examples. All proportions in the examples and elsewhere in the specification are by weight unless specifically stated otherwise.

All documents, e.g., patents and journal articles; cited above or below are hereby incorporated by reference in their entirety. In the following examples, all amounts are stated in percent by weight of active material unless indicated otherwise. One skilled in the art will recognize that modifications may be made in the invention without deviating from the spirit or scope of the invention. The invention is illustrated further by the following examples which are not to be construed as limiting the invention or scope of the specific procedures or compositions described herein. All levels and ranges, temperatures, results etc., used herein are approximations unless otherwise specified.

### EXAMPLES

### Procedure for Making Soap/SME (Sulfonated Methyl Ester) Combars

One procedure for making soap/SME combars is as follows:
(1) Neat soap is melted in a steam jacketed crutcher (18-200° F.)
(2) alpha sulfomethyl ester, as a dried paste or an aqueous solution, is added to the crutcher with stirring, and agitation contained for 5 minutes
(3) Additives to reduce tackiness, such as glycerine or sodium chloride (0.1 to 2.0%) can be introduced into the crutcher at this point and stirring continued for another 2 minutes.
(4) The wet soap is air-dried or vacuum-dried to reduce the moisture level to below 5%.
(5) To milled soap chips, perfume, titanium dioxide and other minor additives are added and milled again (this time with the crimper plate in position)
(6) The soap mix is processed through a Beck plodder (Stephan Beck Plodder Co). The temperature of the plodder is maintained at 90-100° F. using a water circulation system
(7) Bars are pressed from the extruded ribbon using a Midget Multipress (Denison Co) equipped with a standard rectangular die

### Example #1

### Monosalt Sulfonated Methyl Ester (SME) MC-48 Preparation

MC-48 as defined above is commercially available from a variety of sources. Its method of manufacture is well known to those skilled in the art.

### Example #2

### Disalt Sulfonated Fatty Acid (SFA) Preparation

Approximately 3500 grams of MC-48 acid is placed in a 4 L beaker and with rapid agitation, approximately 330 grams of sodium hydroxide is added slowly. Upon complete addition of the sodium hydroxide, the resulting SFA material had a thick, pasty consistency. The crude SFA is re-crystallized by washing with methanol, water and salting out the purified SFA product. The crude SFA is analyzed by titrating the material with 0.02N hyamine, which indicated that approximately 46.6% disodium salt of MC-48 is present. The recrystallized SFA product is approximately 99.8% disodium salt of MC-48.

### Example #3

### 1:1 Ratio of SME to SFA Sample Preparation

Approximately 138.5 grams of MC-48 acid is added to a 1L resin kettle, equipped with heating means, agitation means, pH measurement means and a nitrogen sweep. The acid is heated to 55° C. and approximately 18.7 g of sodium hydroxide powder is added in small portions. As the sodium hydroxide is added an exotherm of 55° C. to about 71° C. occurred, during which time cooling is provided to keep the mixture below approximately 80° C. Near the end of the sodium hydroxide addition, the mixture became very thick and approximately 15.6 grams of methanol is added to keep the mixture semi-fluid. The final product is a paste at room temperature, i.e. 25° C. The final SFA/SME product is titrated with 0.02N hyamine which showed the material to be approximately 41.65% SME (mono salt) and approximately 40.34% SFA (disalt).

### Example #4

### 2:1 Ratio SME to SFA Sample Preparation

Approximately 53.4 grams of undigested a-sulfomethyl ester acid is placed in a 500 mL 4-neck flask, equipped with a heating means, a condenser and stirring means. The acid is heated to 130° C. for 1 minute to digest the acid. The acid is cooled after digestion to 75° C., and approximately 5.3 grams of anhydrous methanol is added, which produced an exotherm to approximately 85° C. Next, approximately 6.4 grams hydrogen peroxide (35% soln.) is added and the resulting mixture heated to about 120° C. for about 5 minutes. After this period of time, the mixture is cooled to about 60° C. and 8.82 grams water is added, producing a gel-like mixture. The mixture is then further cooled to 40° C. and sodium hydroxide (50% soln.) is added dropwise until a pH of 6 is achieved. The final product is a soft, flowable, yellow gel. The actives are determined, via titration with 0.02N hyamine, to be 46.3% SME (monosalt) and 22.5 SFA (disalt).

### Example #5

### 25:1 Ratio SME to SFA Sample Preparation

Approximately 50 grams of undigested a-sulfomethyl ester acid is placed in a 500 mL round bottom flask and heated to 130° C. for 1 minute using a hot oil bath. A mechanical stirrer with a glass shaft and teflon blade is used to ensure thorough mixing. The apparatus included a condenser (allihn type) to prevent loss of any solvent vapors. The acid is cooled after digestion to 70° C., and approximately 5.3 grams of anhydrous methanol is added and thoroughly combined. This is followed by the addition of approximately 1.825 grams hydrogen peroxide (50% soln.) and heating of the resulting mixture to about 89° C. for about 64 minutes. After this period of time, the mixture is cooled to about 40° C. and 64.7 grams water is added and mixed thoroughly. The acid is neutralized by the dropwise addition of sodium hydroxide (50% soln) until a pH of about 6.5 is achieved, all the while maintaining the temperature below 45° C. using a water/ice bath. The final product is analyzed by titration with 0.02N hyamine, and found to comprise 35.82% SME (monosalt) and 1.36 SFA (disalt), with the SME:SFA ratio being 26.3:1.

### Example #6

### Preparation of Samples Containing Various Amounts of SME and SFA

In general, samples containing differing amounts of SFA and SME (e.g., total amounts of each or either present in the mixture, and optionally present with respect to varying amounts of total SFA and SME actives) can be obtained, for instance, by varying the hydrolysis of SME to SFA (e.g., by varying hydrolysis conditions, and/or amount of methanol applied for hydrolysis). Similarly, mixtures can be combined, and/or varying amounts of either pure (or relatively pure) SME or SFA can be added to adjust the concentration of a particular mixture. One skilled in the art would easily know how to obtain the particular ratios referenced herein (if not otherwise disclosed) as well as further ratios and formulations encompassed by the scope of the invention.

### Example #7

### Preparation of Toilet Bar

Tables 2a-d provide examples of skin cleansing combo toilet bars, which provide improved skin mildness, while maintaining desirable soap bar properties (e.g. effective skin cleanser and good aesthetics):

### Tables 2a-d. Examples of Alpha Step BSS-45 Based Combo Bar Formulations

### Active Basis in Finished Bars, %

TABLE 2a

| Components | Example 1 | Example 2 | Example 3 | Example 4 |
|---|---|---|---|---|
| Tallow/coco soap | 75.8 | 69.8 | 67.8 | 63.9 |
| (85/15) | | | | |
| ALPHA STEP BSS- | 7.5 | 7.5 | 7.5 | 15.0 |
| 45 ® (1) | | | | |
| Coconut Fatty Acids | 1.0 | 6.0 | 8.0 | 2.0 |
| Glycerine | 1.0 | 2.0 | 2.0 | 3.5 |
| Sodium Chloride | 0.5 | 0.5 | 0.5 | 1.4 |
| Water | 10.0 | 10.0 | 10.0 | 10.0 |
| Fragrance | 1.2 | 1.2 | 1.2 | 1.2 |
| Minor additives | 3.0 | 3.0 | 3.0 | 3.0 |
| (colorants, | | | | |
| Antioxidants, EDTA, | | | | |
| fillers, etc) | | | | |
| TOTAL | 100.0 | 100.0 | 100.0 | 100.0 |

TABLE 2b

| Components | Example 5 | Example 6 | Example 7 | Example 8 |
|---|---|---|---|---|
| Tallow/coco soap | 61.9 | 60.3 | 52.8 | 51.3 |
| (85/15) | | | | |
| ALPHA STEP BSS- | 15.0 | 15.0 | 15.0 | 20.0 |
| 45 ® (1) | | | | |
| Coconut Fatty Acids | 4.0 | 6.0 | 10.0 | 10.0 |
| Glycerine | 3.5 | 3.5 | 7.0 | 4.0 |
| Sodium Chloride | 1.4 | 1.0 | 1.0 | 1.0 |
| Water | 10.0 | 10.0 | 10.0 | 10.0 |
| Fragrance | 1.2 | 1.2 | 1.2 | 1.2 |
| Minor additives | 3.0 | 3.0 | 3.0 | 3.0 |
| (colorants, | | | | |
| Antioxidants, EDTA, | | | | |
| fillers, etc) | | | | |
| TOTAL | 100.0 | 100.0 | 100.0 | 100.0 |

TABLE 2c

| | Ex. | Ex. | Ex. | Ex. | Ex. | Ex. | Ex. |
|---|---|---|---|---|---|---|---|
| Components | 9² | 9A³ | 10² | 10A³ | 11² | 11A³ | 11B⁴ |
| Tallow/coco | 60.3 | 60.3 | 60.3 | 60.3 | 55.3 | 55.3 | 65.3 |
| soap | | | | | | | |
| (85/15) | | | | | | | |
| ALPHA STEP | 12.0 | 12.0 | 10.0 | 10.0 | 15.0 | 15.0 | 6.7 |
| BSS-45 ® (1) | | | | | | | |
| Ninol ® | 3.0 | 3.0 | 5.0 | 5.0 | 5.0 | 5.0 | 3.3 |
| Stearic/Coconut | 6.0 | 6.0 | 6.0 | 6.0 | 6.0 | 6.0 | 6.0 |
| Fatty | | | | | | | |
| Acids | | | | | | | |
| Glycerine | 3.5 | 3.5 | 3.5 | 3.5 | 3.5 | 3.5 | 3.5 |
| salt | 1.0 | 1.0 | 1.0 | 1.0 | 1.0 | 1.0 | 1.0 |
| Water | 10.0 | 10.0 | 10.0 | 10.0 | 10.0 | 10.0 | 10.0 |
| Fragrance | 1.2 | 1.2 | 1.2 | 1.2 | 1.2 | 1.2 | 1.2 |
| Minor | 3.0 | 3.0 | 3.0 | 3.0 | 3.0 | 3.0 | 3.0 |
| additives | | | | | | | |
| (colorants, | | | | | | | |
| Antioxidants, | | | | | | | |
| EDTA, | | | | | | | |
| fillers, | | | | | | | |
| etc) | | | | | | | |
| TOTAL | 100.0 | 100.0 | 100.0 | 100.0 | 100.0 | 100.0 | 100.0 |

TABLE 2d

| | Example | Example | Example | Example |
|---|---|---|---|---|
| Components | 12 | 13 | 14 | 15 |
| Tallow/coco soap | 81.3 | 66.3 | 64.8 | 69.8 |
| (80/20) | | | | |
| ALPHA STEP BSS- | 0.0 | 15.0 | 15.0 | 15.0 |
| 45 ® (1) | | | | |
| Coconut Fatty Acids | 4.0 | 4.0 | 4.0 | 4.0 |
| Glycerine | 3.5 | 3.5 | 5.0 | 0.0 |
| Sodium Chloride | 1.0 | 1.0 | 1.0 | 1.0 |
| Water | 10.0 | 10.0 | 10.0 | 10.0 |
| Minor additives | 0.2 | 0.2 | 0.2 | 0.2 |
| (Citric Acid, EDTA) | | | | |
| TOTAL | 100.0 | 100.0 | 100.0 | 100.0 |

TABLE 2e

| | Example | Example | Example |
|---|---|---|---|
| Components | 16 | 17 | 18 |
| Tallow/coco soap | 56.8 | 64.8 | 40.8 |
| (85/15) | | | |
| ALPHA STEP BSS- | 14.0 | 7.0 | 7.0 |
| 45 ® (1) | | | |
| NINOL | 10.0 | 3.0 | 3.0 |
| Stearic/Coconut Fatty | 1.0 | 2.0 | 2.0 |
| Acids | | | |
| Glycerine | 3.0 | 2.0 | 10.0 |
| Sodium Chloride | 1.0 | 1.0 | 1.0 |
| Water | 10.0 | 16 | 10.0 |
| Fragrance | 1.2 | 1.2 | 1.2 |
| Minor additives | 3.0 | 3.0 | 3.0 |
| (colorants, | | | |
| antioxidants, EDTA, | | | |
| fillers, etc.) | | | |
| TOTAL | 100.0 | 100.0 | 100.0 |

(1) Stepan ™ Coconut Sodium Alpha Sulfo Methyl Ester 1:1 Mono/di ratio from Stepan Co.
(2) Stepan ™ Ninol ® LMP (LMP: Lauryl Monoethanolamide); salt is sodium chloride
(3) Stepan ™ Ninol ® CMP (CMP: Coconut Monoethanolamide); salt is sodium chloride
(4) Salt is 1:1 sodium chloride:magnesium sulfate

The compositions above are prepared in substantially the same way. Below is the manufacturing procedure for a typical formulation (example No. 10, in this example):

Crutching Step. About 127.3 parts of a mix containing: 31.67% water, 46.7% 85/15 tallow/coconut (T/CN) soap, 0.43% Sodium chloride, 2.75% glycerine, 4.69% coconut free fatty acid (CNFA), 9.46% of sodium coconut alpha sulfo Metyl ester 1:1 Mono/di ratio paste, and 3.93% of Ninol CMP or LMP are added to a crutcher in the indicated order. Mix the product at 85 to 90° C.

Vacuum Drying Step. The crutcher mix is vacuum dried at approximately 50 mm Hg absolute pressure to reduce the moisture content of the mix to 10% and to plod this soap into noodles.

Amalgamating Step. The soap noodles are weighed and placed in a batch amalgamator. To about 97.0 parts noodles in the amalgamator are added: 0.50 part TiO₂, 2.0 parts perfume, 0.1% BHT, 0.1% Citric Acid, 0.15 part colorant solution, and 0.15 part of a solution which contains ca. 40% EDTA. The combined ingredients are mixed thoroughly.

Milling Step. Three-roll soap mills are set up with all rolls at 85° C.-105° F. (29° C.-41° C.). The mixture from the amalgamator is passed through the mills several times to obtain a homogeneous mix. This is an intimate mixing step.

Plodding and Stamping Steps. A conventional plodder is set up with the barrel temperature at about 35° C. and the nose temperature at about 42° C. The plodder used is a dual stage twin screw plodder that allows for a vacuum of about 40 to 65 mm Hg between the two stages. The soap log extruded from the plodder is typically round, and is cut into individual plugs. These plugs are then stamped on a conventional soap stamping apparatus to yield the finished toilet soap bar.

It has been discovered that the soap bars made from the above compositions possess surprising performance and processing advantages. These advantages are demonstrated below by the marring data, phase behavior and rheology profile.

Soap Bar Marring Data

Marring is the damage incurred by impact to a soap bar during handling and shipping. It is a well-known characteristic by which consumers rate a bar. Bar soap manufacturers prefer a soap formulation with low mar characteristics to reduce consumer rejection should the bars incur any damage or rough handling during shipping. The bars of the invention show little damage when dropped compared to commercial combo bars. As an illustration of this, soap bars prepared according to the invention are subjected to a test that quantitatively compares different bars by their marring characteristics.

### Bar Marring Test Method

Each sample is weighed and then dropped from a specific height to mar the bars. It was determined that exactly 7 feet would provide an extreme enough impact to clearly determine the marring characteristics of the bars. The bars would be dropped in a way that the small end of the bar would strike the ground to provide the most visible damage possible (striking perpendicular to the extrusion of the bars). The bars are then analyzed for their level of damage in the form of a dry-impact bar cracking scale. using this scale the mar value of the bar is determined through ranking of the visible damage to the bar. (see Table 3).

TABLE 3
The Dry-Impact Cracking Scale

| Mar Value | Visible characteristics. |
|---|---|
| 0 | No cracks or chips, a smooth dent |
| 1 | Very fine spider cracks |
| 2 | Hair-line fracturing |
| 3 | Visible deep cracks with potential for chipping |
| 4 | Slight chipping along edge of damage |
| 5 | Noticeable chips from around area of impact |
| 6 | Obvious deforming/shattering of bar, |
| | large chunks broken off of bar. |

The bar mar test method was analyzed for reproducibility. Samples are tested in triplicate to ensure reproducibility and determine the standard deviation. The average standard deviation of the mar values for the samples is 0.39, showing a high reproducible rate within a range of 1 on the dry-impact cracking scale (see Table 3).

The test method is used to determine the marring characteristics of several inventive trial bars and several commercial bars. Each bar is dropped from a 7 foot height and the damage measured to calculate the total marring value of each sample.

The results summarized in Table 4 indicate that the inventive trial bars show a marring value of zero, which is lower than any of the commercial combo bars evaluated in the test. It is apparent that the inventive compositions provides a bar with lower mar than the commercial plain soap and combo bars.

TABLE 4
Marring Test Results

| | Mar Mean |
|---|---|
| Sample | Value |
| Commercial US Combo Bar A | 4.66 |
| Commercial US Combo Bar B | 3.33 |
| Commercial Mexican Combo Bar | 1.66 |
| Example 3, Table 2a. | 0.33 |
| Example 5, Table 2b. | 0.0 |
| Example 6, Table 2b. | 0.0 |

### Phase Behavior

The following examples relate to the phase behavior and rheology profiles of SME soap slurries of the invention. The example information is given in Table 5. Sample “control” (Example 12) is the neat soap bar material without SME and functions as a control. Examples 13-15 are Stepan SME bar slurries with various concentrations of glycerine. Both Control and Stepan SME slurries contained 32% moisture level:

TABLE 5
Example Information of SME Soap Slurry

| | | Glycerine | SME |
|---|---|---|---|
| Example | Major Composition | (%) | (%) |
| Control | 80:20 Tallow/Coco | 3.5 | 0 |
| Example 12 | Soap + Glycerine | | |
| Example 13 | SME + Soap + Glycerine | 3.5 | 15 |
| Example 14 | SME + Soap + Glycerine | 5.0 | 15 |
| Example 15 | SME + Soap | 0 | 15 |

Phase behavior was studied using a cross-polarized microscope (Olympus) equipped with a hot stage (Instec). The sample was spread on and then sealed between a glass slide and a cover glass at room temperature. By doing this, the concentration of the sample was maintained constant as moisture is locked in. The phase behavior of a soap bar material was obtained by analyzing its texture. During the texture observation, the sealed sample was kept at designated temperature for at least 10 minutes before the analysis.

Texture is the image of a material under microscope, and it can be directly related to the particle arrangement in a sample. Different particle arrangement results in different phases. For example, if particles align into two-dimensional layers, the material is in a lamellar phase. Particle arrangement depends strongly on sample environment. When the sample concentration, temperature or solvent change, particle arrangement will also change to adapt the new environment. Therefore, changes from one state to another can be monitored through the texture transition.

The phase behavior of four soap bar slurries in a temperature range from 30° C. to 95° C. is investigated. It was found that the phase transition temperature is very different for the four samples. Also, at a given temperature, 70° C., the texture of one sample is different from the other. The results are summarized in Table 6.

TABLE 6
Phase Behavior of SME Soap Slurries

| | Control | Example | Example | Example |
|---|---|---|---|---|
| | (Example 12) | 13 | 14 | 15 |
| Phase at | Hexagonal | Lamellar | Lamellar/ | Hexagonal |
| 70° C. | | | isotropic | |
| Texture at | Hexagonal | Maltese | Maltese | Hexagonal |
| 70° C. | gel | crosses | crosses | gel |
| | | and oily | (lamellar) | |
| | | streak (lamellar) | | |
| Phase | ˜80° C. | ˜60° C. | ˜60° C. | ˜90° C. |
| Transition | | | | |
| Temperature | | | | |
| (hexagonal | | | | |
| change to | | | | |
| lamellar) | | | | |

At 30° C., all four soap bar slurries are heavy pastes. Their texture is not very characteristic. The non-characteristic texture might be caused by the interference of shear induced birefringence during sample preparation. The control's texture does not change much until the temperature is increased to 80° C. At 80° C., the texture slowly change to mosaic type, indicating the material is in a lamellar phase. Although the soap is in a lamellar phase at this temperature, no Maltese cross or oily streaks can be found. At 85° C., the liquid crystal phase abruptly changed to an isotropic liquid.

The texture of Example 13 changes dramatically when the temperature is increased to 60° C. Typical lamellar and hexagonal textures can be clearly observed at this temperature. The relatively fast change in texture indicating that the particles can easily reorient themselves. When the temperature rises to 70° C., the material turns into a complete lamellar phase with distinctive Maltese cross and oily streaks texture. The texture changes very fast and some flow paths can be observed.

The differences between Example 13 formulation and “control” (example 12) formulation in phase behavior clearly demonstrated SME is crucial in determining particle arrangement in a soap material. With its existence, molecules are much easier to align into layers and lamellar phase can be attained at a much lower temperature. However, SME alone without glycerine does not demonstrate such a function, because the phase transition of Example 15 occurred at −90° C. Therefore, the combination of SME with glycerine is preferred for generating a lamellar phase for Example 13 formulation at relatively lower temperature.

Rheology Profile

It is known that in a lamellar phase particles are arranged into layers. Because particles can slide between layers in this structure, it is much easier to move them than to move the particles arranged in cubic or hexagonal pattern. Therefore, lamellar phase usually has much lower viscosity than the other types of liquid crystalline phases, and is much easier to process.

Soap bar materials containing SME and glycerine easily arrange themselves into a lamellar phase. It has been found that these materials are easy to process. As support for this finding, the rheology of the four soap slurries is studied.

Rheology measurements are done with a Rheolyst AR1000 rheometer (TA Instrument). A 4 cm stainless steel plate with solvent trap is used in the plate—plate configuration. Water is filled in the solvent trap for maintaining moisture. The gap between plates is 100 μm.

For a continuous flow test, the sample is heated up to 70° C. and equilibrated at this temperature for 1 minute before shear is applied. The shear rate is kept constant at 2 l/S. For a stepped shear flow, a sample is kept at 70° C. for 3 minutes before the measurement is taken. The shear rate is increased linearly from 0.2 l/S to 5 l/S.

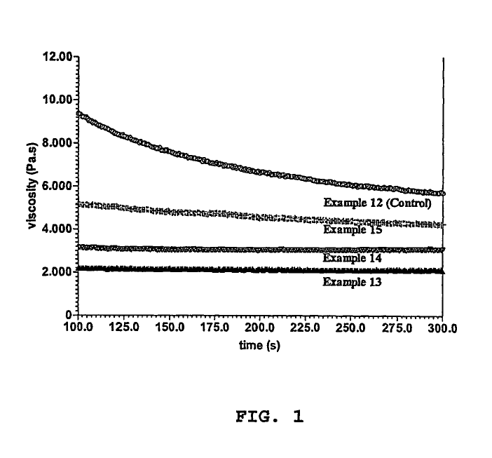

The viscosity of the soap bar materials is obtained from constant shear flow measurements. The temperature and the shear rate are kept constant during the test. The results are shown in FIG. 1. Example 13 formulation has the lowest viscosity. A constant viscosity is reached after 100 seconds of shearing. The Stepan Example 14 formulation has higher viscosity than Example 13 formulation. It also gets to a stable viscosity very fast. The Example 15 formulation and the Example 12 formulation (control), however, not only have much higher viscosity, but also cannot reach a stable viscosity even after 5 minutes of constant shearing. Some typical viscosity numbers for these materials are listed in Table 7.

TABLE 7
Viscosities of SME Soap Slurries from Constant
Flow Measurement

| | Viscosity at | Viscosity at 300 |
|---|---|---|
| | 100 Sec | Sec |
| Sample | (Pa · S) | (Pa · S) |
| Control | 9.3 | 5.7 |
| (Example 12) | | |
| Example 13 | 2.1 | 2.1 |
| Example 14 | 3.1 | 3.1 |
| Example 15 | 5.1 | 4.3 |

The viscosity results clearly demonstrated that the mixture of SME with glycerine can dramatically reduce the viscosity of a soap slurry. The efficiency in viscosity reduction is also strongly dependent on the amount of SME and glycerine in a sample. From this study, Stepan SME Combo 4 is found to be more efficient than Stepan SME Combo 5. The different speed to reach equilibrium for the four examples suggests that soaps containing SME and glycerine are in a lamellar phase at 70° C., while the other two materials are not, because it takes a much longer time for a non-lamellar phase to align in a shear field.

The differences in rheology for the four soap slurries are also supported by their yield stress and thixotropy measurements. Yield stress is obtained from extrapolating a stepped flow curve to zero shear rate. The thixotropy is calculated from the curve fitting using Casson's equation. The values are given in Table B. At 70° C., all of the soap bar materials exhibited a yield stress and showed strong thixotropic behavior. Their viscosity decreased very rapidly with increase of shear rate. The strong thixotropic behavior of “control” (Example 12 formulation) indicates more structure has been broken down during the shearing process, leading to more particles have to orient themselves into a lined structure. While under the same conditions, the re-orientation requirement is much less for the soap slurry with SME and glycerine.

TABLE 8
Yield Stress and Thixotropy of SME Soap Slurries

| | Yield Stress | |
|---|---|---|
| Example | (Pa) | Thixotropy |
| Control | 45 | 268 |
| (Example | | |
| 12) | | |
| Example 13 | 1.8 | 12.8 |
| Example 14 | 3.5 | 21.1 |
| Example 15 | 14 | 61.2 |

The data obtained in this study and the conclusions drawn from the work clearly suggest that both SME and glycerine in desired amount are necessary to achieve a product with significantly lower phase transition temperature (−60° C.). At this temperature, Example 13 formulation goes into lamellar phase, which has significantly lower viscosity and requires very low yield stress, resulting in much easier mixing, more efficient heat transfer, and faster drying. In the absence of either SME or glycerine, the phase transition temperature is much higher and the material goes into a primarily hexagonal high viscosity phase, which is known to be more difficult to process.

In lamellar structure, water binds with the polar groups of surfactants and form in a sheet type highly ordered structured water phase. The water is distributed more evenly and is available uniformly as its structure recovery under shear is fast. This results into much better drying properties of lamellar soap melt. Due to uniform moisture distribution in the soap melt, there will be very few dry and moist spots in the extruded bars. During storage or use these bars, they may not lose or absorb different amount of water causing the bar to develop cracks at the point of moisture gradient difference. Thus the bar produced from a lamellar soap melt will have much uniform evaporation of water over time and would display characteristics of much better elasticity.

Without being bound by any particular theory, it is believed that the preferred compositions can evenly distribute the bound water and this water is not easily available for evaporation under storage temperatures and as a result very little crystallinity occurs and the bar is less susceptible to marring. This is another positive and desirable attribute of SME soap bar technology.

The invention and the manner and process of making and using it, are now described in such full, clear, concise and exact terms as to enable any person skilled in the art to which it pertains, to make and use the same. It is to be understood that the foregoing describes some embodiments of the invention and that modifications may be made therein without departing from the spirit or scope of the invention as set forth in the claims. To particularly point out and distinctly claim the subject matter regarded as invention, the following claims conclude this specification.

1. A process for preparing a personal cleansing and laundry detergent bar pre-blend, comprising the sequential steps of:
(a) forming at a temperature of about 65° C. to about 105° C. a substantially homogeneous aqueous liquid mixture comprising
1) from about 58% to about 93% by weight of an approximately 70% aqueous soap slurry, the soap being of the formula 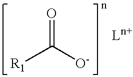
wherein R₃ is a C₆-C₂₂ hydrocarbyl group, an alkyl group, or combination thereof, n is 1 or 2, and L is a cation; and
2) from about 1% to about 15% by weight of a fatty acid of the formula 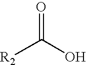
wherein R₂ is a C₆-C₂₂ hydrocarbyl group, an alkyl group, or combination thereof; and
3) from about 2% to about 30% by weight of an approximately 55% aqueous mixture of anionic surfactants, the anionic surfactants comprising:
i) an alpha sulfonated alkyl ester of the formula 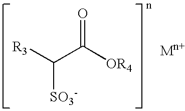
wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
ii) a sulfonated fatty acid of the formula 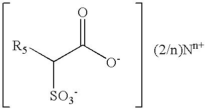
wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
wherein the ratio of i) to ii) is from about 10:1 to about 1:10;
4) from about 0.5% to about 2% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof;
5) from about 0.5% to about 10% by weight of a polyhydridic alcohol; and
6) from 0 to about 10% by weight of an alkanolamide of the formula 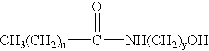
wherein n=6-16, and y is 2-4;
(b) removing from about 5% to about 90% by weight of the total water from the liquid mixture to form a thickened mixture; and
(c) extruding the thickened mixture to form flaked solid or semi-solid particles.

2. A process according to claim 1, further comprising plodding the flaked solid or semi-solid particles to form plodded particles.

3. A process according to Claim 2, further comprising extruding the plodded particles to form a billet, cutting the billet, and stamping the cut billet to yield a personal cleansing or laundry detergent bar.

4. A process according to claim 1, wherein R₁ is a C₆-C₁₈ hydrocarbyl group, an alkyl group, or combination thereof, and M is sodium or potassium, or a mixture thereof.

5. A process according to claim 4, wherein the soap is present from about 68% to about 78% by weight.

6. A process according to claim 1, wherein R₂ is a C₁₂-C₂₀ hydrocarbyl group, an alkyl group, or combination thereof.

7. A process according to claim 6, wherein the fatty acid is present from about 2% to about 7% by weight.

8. A process according to claim 1, wherein R₁₃ is a C₈-C₂₀ hydrocarbyl group, an alkyl group, or combination thereof, R₄ is methyl and M is hydrogen, sodium, potassium, calcium, magnesium ammonium, monoethanolammonium, diethanolammonium, triethanolammonium, or a mixture thereof, R₅ is a C₈-C₂₀ hydrocarbyl group, an alkyl group, or combination thereof, and N is hydrogen, sodium, potassium, calcium, magnesium, ammonium, monoethanolammonium, diethanolammonium, triethanolammonium, or a mixture thereof.

9. A process according to claim 8, wherein the ratio of the mixture of anionic surfactants i:ii is from about 3:1 to about 1:3.

10. A process according to claim 1, wherein the salt is sodium chloride.

11. A process according to claim 1, wherein the polyhydridic alcohol is selected from the group consisting of glycerine, polyglycerol esters, sorbitol and propylene glycol, or a mixture thereof.

12. A process according to claim 11, wherein the polyhydridic alcohol is glycerine.

13. A process according to claim 1, wherein removing the water from the liquid mixture is accomplished by scraped wall vacuum evaporation drying under reduced pressure or heated drum drying at ambient pressure.

14. A process according to claim 13, wherein about 55% to about 85% by weight water is removed from the liquid mixture.

15. A process according to claim 13, wherein about 60% to about 80% by weight water is removed from the liquid mixture.

16. A personal cleansing and laundry detergent bar pre-blend, produced by the process of claim 1.

17. A personal cleansing or laundry detergent bar produced by the process of claim 3.

18. A process for preparing a personal cleansing and laundry detergent bar pre-blend, comprising the sequential steps of:
(a) forming at a temperature of about 65° C. to about 105° C. a substantially homogeneous aqueous soap-fatty acid liquid mixture comprising
1) from about 58% to about 93% by weight of an approximately 70% aqueous soap slurry, the soap being of the formula 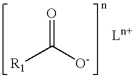
wherein R₁ is a C₆-C₂₂ hydrocarbyl group, an alkyl group, or combination thereof, n is 1 or 2, and L is a cation; and
2) from about 1% to about 15% by weight of a fatty acid of the formula 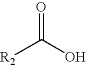
wherein R₂ is a C₆-C₂₂ hydrocarbyl group, an alkyl group, or combination thereof; and
(b) adding to the soap-fatty acid liquid mixture to form a first intermediate liquid mixture at a temperature of about 65° C. to about 105° C.
1) from about 0.5% to about 2% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof;
2) from about 0.5% to about 5.0% by weight of a polyhydridic alcohol; and
3) from 0 to about 10% by weight of an alkanolamide of the formula 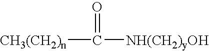
wherein n=6-16, and y is 2-4;
(c) adding to the first intermediate liquid mixture to form a second intermediate liquid mixture at a temperature of about 65° C. to about 105° C. from about 2% to about 30% by weight of an approximately 55% aqueous mixture of anionic surfactants, the anionic surfactants comprising
i) an alpha sulfonated alkyl ester of the formula 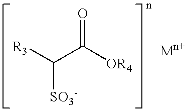
wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
ii) a sulfonated fatty acid of the formula 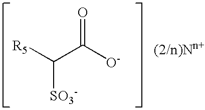
wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
wherein the ratio of i) to ii) is from about 10:1 to about 1:10;
(d) removing from about 50% to about 90% by weight of the total water from the second intermediate liquid mixture to form a thickened mixture; and
(e) extruding the thickened mixture to form flaked solid or semi-solid particles.

19. A process according to claim 18, further comprising plodding the flaked solid or semi-solid particles to form plodded particles.

20. A process according to claim 19, further comprising extruding the plodded particles to form a billet, cutting the billet, and stamping the cut billet to yield a personal cleansing or laundry detergent bar.

21. A process according to claim 18, wherein R₁ is a C₆-C₁₈ hydrocarbyl group, an alkyl group, or combination thereof, and M is sodium or potassium, or a mixture thereof.

22. A process according to claim 21, wherein the soap is present from about 68% to about 78% by weight.

23. A process according to claim 18, wherein R₂ is a C₁₂-C₂₀ hydrocarbyl group, an alkyl group, or combination thereof.

24. A process according to claim 23, wherein the fatty acid is present from about 2% to about 7% by weight.

25. A process according to claim 18, wherein R₃ is a C₈-C₂₀ hydrocarbyl group, an alkyl group, or combination thereof, R₄ is methyl and M is hydrogen, sodium, potassium, calcium, magnesium ammonium, monoethanolammonium, diethanolammonium, triethanolammonium, or a mixture thereof, R₅ is a C₈-C₂₀ hydrocarbyl group, an alkyl group, or combination thereof, and N is hydrogen, sodium, potassium, calcium, magnesium, ammonium, monoethanolammonium, diethanolammonium, triethanolammonium, or a mixture thereof.

26. A process according to claim 25, wherein the ratio of the mixture of anionic surfactants is from about 3:1 to about 1:3.

27. A process according to claim 18, wherein the salt is sodium chloride.

28. A process according to claim 27, wherein the polyhydridic alcohol is selected from the group consisting of glycerine, polyglycerol esters, sorbitol and propylene glycol, or a mixture thereof.

29. A process according to claim 28, wherein the polyhydridic alcohol is glycerine.

30. A process according to claim 18, wherein removing the water from the liquid mixture is accomplished by scraped wall vacuum evaporation drying under reduced pressure or heated drum drying at ambient pressure.

31. A process according to claim 30, wherein about 55% to about 85% by weight of the water is removed from the liquid mixture.

32. A process according to claim 31, wherein about 60% to about 80% by weight of the water is removed from the liquid mixture.

33. A personal cleansing and laundry detergent bar pre-blend, produced by the process of claim 18.

34. A personal cleansing or laundry detergent bar produced by the process of claim 20.

35. A process for preparing a personal cleansing and laundry detergent bar pre-blend, comprising the sequential steps of:
(a) forming at a temperature of about 65° C. to about 105° C. a substantially homogeneous aqueous soap-fatty acid liquid mixture comprising
1) from about 58% to about 93% by weight of an approximately 70% aqueous soap slurry, the soap being of the formula 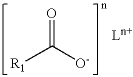
wherein R₃ is a C₆-C₂₂ hydrocarbyl group, an alkyl group, or combination thereof, n is 1 or 2, and L is a cation; and
2) from about 1% to about 15% by weight of a fatty acid of the formula 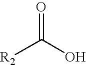
wherein R₂ is a C₆-C₂₂ hydrocarbyl group, an alkyl group, or combination thereof; and
(b) forming at a temperature of about 65° C. to about 105° C. a liquid alcohol-salt-anionic surfactant mixture comprising
1) from about 0.5% to about 2% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof; and
2) from about 0.5% to about 10% by weight of a polyhydridic alcohol;
3) from about 2% to about 30% by weight of an approximately 55% aqueous mixture of anionic surfactants, the anionic surfactants comprising
i) an alpha sulfonated alkyl ester of the formula 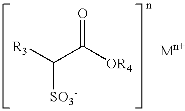
wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
ii) a sulfonated fatty acid of the formula 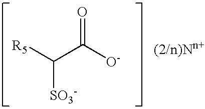
wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
wherein the ratio of i) to ii) is from about 10:1 to about 1:10;
(c) combining said liquid alcohol-salt-anionic surfactant mixture and said liquid soap-fatty acid mixture at a temperature of about 65° C. to about 105° C. to form an intermediate liquid mixture;
(d) optionally adding to said intermediate liquid mixture from 0 to about 10% by weight of an alkanolamide of the formula 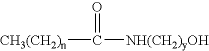
wherein n=6-16, and y is 2-4;
(e) removing from about 50% to about 90% by weight of the total water from the intermediate liquid mixture to form a thickened mixture; and
(f) extruding the thickened mixture to form flaked solid or semi-solid particles.

36. A process according to claim 35, further comprising plodding the flaked solid or semi-solid particles to form plodded particles.

37. A process according to claim 36, further comprising extruding the plodded particles to form a billet, cutting the billet, and stamping the cut billet to yield a personal cleansing or laundry detergent bar.

38. A process according to claim 35, wherein R₁ is a C₆C₁₈ hydrocarbyl group, an alkyl group, or combination thereof, and M is sodium or potassium, or a mixture thereof.

39. A process according to claim 38, wherein the soap is present from about 68% to about 78% by weight.

40. A process according to claim 35, wherein R₂ is a C₁₂-C₂₀ hydrocarbyl group, an alkyl group, or combination thereof.

41. A process according to claim 40, wherein the fatty acid is present from about 2% to about 7% by weight.

42. A process according to claim 35, wherein R₃ is a C₈-C₂₀ hydrocarbyl group, an alkyl group, or combination thereof, R₄ is methyl and M is hydrogen, sodium, potassium, calcium, magnesium ammonium, monoethanolammonium, diethanolammonium, triethanolammonium, or a mixture thereof, R₅ is a C₈-C₂₀ hydrocarbyl group, an alkyl group, or combination thereof, and N is hydrogen, sodium, potassium, calcium, magnesium, ammonium, monoethanolammonium, diethanolammonium, triethanolammonium, or a mixture thereof.

43. A process according to claim 42, wherein the ratio of the mixture of anionic surfactants is from about 3:1 to about 1:3.

44. A process according to claim 35, wherein the salt is sodium chloride.

45. A process according to claim 44, wherein the polyhydridic alcohol is selected from the group consisting of glycerine, polyglycerol esters, sorbitol and propylene glycol, or a mixture thereof.

46. A process according to claim 45, wherein the polyhydridic alcohol is glycerine.

47. A process according to claim 35, wherein removing the water from the liquid mixture is accomplished by scraped wall vacuum evaporation drying under reduced pressure or heated drum drying at ambient pressure.

48. A process according to claim 47, wherein about 55% to about 85% by weight of the water is removed from the liquid mixture.

49. A process according to claim 48, wherein about 60% to about 80% by weight of the water is removed from the liquid mixture.

50. A personal cleansing and laundry detergent bar pre-blend, produced by the process of claim 35.

51. A personal cleansing or laundry detergent bar produced by the process of claim 38.

52. A process for preparing a personal cleansing and laundry detergent bar pre-blend, comprising the sequential steps of:
(a) forming at a temperature of about 65° C. to about 105° C. a substantially homogeneous aqueous soap-fatty acid-anionic surfactant liquid mixture comprising
1) from about 58% to about 93% by weight of an approximately 70% aqueous soap slurry, the soap being of the formula 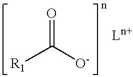
wherein R₁ is a C₆-C₂₂ hydrocarbyl group, an alkyl group, or combination thereof, n is 1 or 2, and L is a cation; and
2) from about 1% to about 15% by weight of a fatty acid of the formula 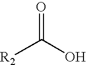
wherein R₂ is a C₆-C₂₂ hydrocarbyl group, an alkyl group, or combination thereof; and
3) from about 2% to about 15% by weight of an approximately 55% aqueous mixture of anionic surfactants, the anionic surfactants comprising
i) an alpha sulfonated alkyl ester of the formula 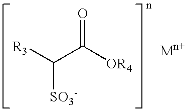
wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
ii) a sulfonated fatty acid of the formula 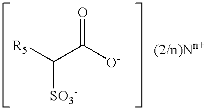
wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
wherein the ratio of i) to ii) is from about 10:1 to about 1:10;
(b) forming at a temperature of about 65° C. to about 105° C. a liquid alcohol-salt-anionic surfactant mixture comprising
1) from about 0.5% to about 2% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof; and
2) from about 0.5% to about 10% by weight of a polyhydridic alcohol;
3) from about 3% to about 15% by weight of an approximately 55% aqueous mixture of anionic surfactants, the anionic surfactants comprising
i) an alpha sulfonated alkyl ester of the formula 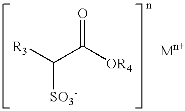
wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
ii) a sulfonated fatty acid of the formula 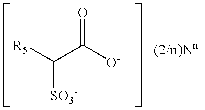
wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
wherein the ratio of i) to ii) is from about 10:1 to about 1:10;
(c) combining said liquid soap-fatty acid-anionic surfactant mixture and said liquid alcohol-salt-anionic surfactant mixture at a temperature of about 65° C. to about 105° C. to form an intermediate liquid mixture;
(d) optionally adding to said intermediate liquid mixture from 0 to about 10% by weight of an alkanolamide of the formula 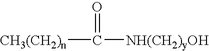
wherein n=6-16, and y is 2-4;
(e) removing from about 50% to about 90% by weight of the total water from the intermediate liquid mixture to form a thickened mixture; and
(f) extruding the thickened mixture to form flaked solid or semi-solid particles.

53. A process according to claim 52, further comprising plodding the flaked solid or semi-solid particles to form plodded particles.

54. A process according to claim 53, further comprising extruding the plodded particles to form a billet, cutting the billet, and stamping the cut billet to yield a personal cleansing or laundry detergent bar.

55. A process according to claim 52, wherein R₁ is a C₆-C₁₈ hydrocarbyl group, an alkyl group, or combination thereof, and M is sodium or potassium, or a mixture thereof.

56. A process according to claim 55, wherein the soap is present from about 68% to about 78% by weight.

57. A process according to claim 52, wherein R₂ is a C₁₂-C₂₀ hydrocarbyl group, an alkyl group, or combination thereof.

58. A process according to claim 57, wherein the fatty acid is present from about 2% to about 7% by weight.

59. A process according to claim 52, wherein R₃ is a C₈-C₂₀ hydrocarbyl group, an alkyl group, or combination thereof, R₄ is methyl and M is hydrogen, sodium, potassium, calcium, magnesium ammonium, monoethanolammonium, diethanolammonium, triethanolammonium, or a mixture thereof, R₅ is a C₈-C₂₀ hydrocarbyl group, an alkyl group, or combination thereof, and N is hydrogen, sodium, potassium, calcium, magnesium, ammonium, monoethanolammonium, diethanolammonium, triethanolammonium, or a mixture thereof.

60. A process according to claim 59, wherein the ratio of the mixture of anionic surfactants is from about 3:1 to about 1:3.

61. A process according to claim 52, wherein the salt is sodium chloride.

62. A process according to claim 61, wherein the polyhydridic alcohol is selected from the group consisting of glycerine, polyglycerol esters, sorbitol and propylene glycol, or a mixture thereof.

63. A process according to claim 62, wherein the polyhydridic alcohol is glycerine.

64. A process according to claim 52, wherein removing the water from the liquid mixture is accomplished by scraped wall vacuum evaporation drying under reduced pressure or heated drum drying at ambient pressure.

65. A process according to claim 64, wherein about 55% to about 85% by weight of the water is removed from the liquid mixture.

66. A process according to claim 65, wherein about 60% to about 80% by weight of the water is removed from the liquid mixture.

67. A personal cleansing and laundry detergent bar pre-blend, produced by the process of claim 52.

68. A personal cleansing or laundry detergent bar produced by the process of claim 54.

69. A composition suitable for formation into precursor cleansing/laundry bar soap noodles, personal cleansing bars and laundry detergent bars comprising:
(a) from about 58% to about 93% by weight of an approximately 70% aqueous soap slurry, the soap being of the formula 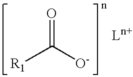
wherein R₁ is a C₆-C₂₂ hydrocarbyl group, an alkyl group, or combination thereof, n is 1 or 2, and L is a cation; and
(b) from about 1% to about 15% by weight of a fatty acid of the formula 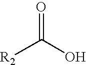
wherein R₂ is a C₆-C₂₂ hydrocarbyl group, an alkyl group, or combination thereof; and
(c) from about 2% to about 30% by weight of an approximately 55% aqueous mixture of anionic surfactants, the anionic surfactants comprising
i) an alpha sulfonated alkyl ester of the formula 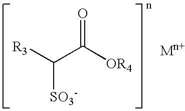
wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
ii) a sulfonated fatty acid of the formula 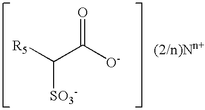
wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
wherein the ratio of i) to ii) is from about 10:1 to about 1:10;
(d) from about 0.5% to about 2% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof;
(e) from about 0.5% to about 5.0% by weight of a polyhydridic alcohol; and
(f) from 0 to about 10% by weight of an alkanolamide of the formula 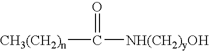
wherein n=6-16, and y is 2-4.

70. A composition according to claim 69, further comprising from about 1% to about 5% by weight paraffin.

71. A composition according to claim 70, wherein R₁ is a C₆-C₁₈ hydrocarbyl group, an alkyl group, or combination thereof, and M is sodium or potassium, or a mixture thereof.

72. A composition according to claim 71, wherein the soap is present from about 68% to about 78% by weight.

73. A composition according to claim 69, wherein R₂ is a C₁₂-C₂₀ hydrocarbyl group, an alkyl group, or combination thereof.

74. A composition according to claim 73, wherein the fatty acid is present from about 2% to about 7% by weight.

75. A composition according to claim 69, wherein R₃ is a C₈-C₂₀ hydrocarbyl group, an alkyl group, or combination thereof, R₄ is methyl and M is hydrogen, sodium, potassium, calcium, magnesium ammonium, monoethanolammonium, diethanolammonium, triethanolammonium, or a mixture thereof, R₅ is a C₈-C₂₀ hydrocarbyl group, an alkyl group, or combination thereof, and N is hydrogen, sodium, potassium, calcium, magnesium, ammonium, monoethanolammonium, diethanolammonium, triethanolammonium, or a mixture thereof.

76. A composition according to claim 75, wherein the ratio of the mixture of anionic surfactants is from about 3:1 to about 1:3.

77. A composition according to claim 69, wherein the salt is sodium chloride.

78. A composition according to claim 69, wherein the polyhydridic alcohol is selected from the group consisting of glycerine, polyglycerol esters, sorbitol and propylene glycol, or a mixture thereof.

79. A composition according to claim 78, wherein the polyhydridic alcohol is glycerine.

80. A personal cleansing/laundry detergent bar comprising:
(a) from about 50% to about 85% by weight of a soap of the formula 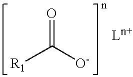
wherein R₁ is a C₆-C₂₂ hydrocarbyl group, an alkyl group, or combination thereof, n is 1 or 2, and L is a cation; and
(b) from about 1% to about 15% by weight of a fatty acid of the formula 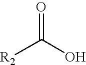
wherein R₂ is a C₆-C₂₂ hydrocarbyl group, an alkyl group, or combination thereof; and
(c) from about 3.5% to about 20% by weight of a mixture of anionic surfactants comprising
i) an alpha sulfonated alkyl ester of the formula 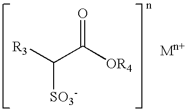
wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and
ii) a sulfonated fatty acid of the formula 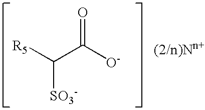
wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof;
wherein the ratio of i) to ii) is from about 10:1 to about 1:10;
(d) from about 0.7% to about 3% by weight of a salt selected from the group consisting of sodium sulfate, sodium chloride, sodium carbonate, potassium sulfate, potassium chloride, potassium carbonate, calcium sulfate, calcium chloride, calcium carbonate, magnesium sulfate, magnesium chloride, or magnesium carbonate, or a mixture thereof;
(e) from about 0.5% to about 6% by weight of a polyhydridic alcohol;
(f) from 0 to about 10% by weight of an alkanolamide of the formula 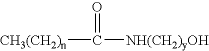
wherein n=6-16, and y is 2-4; and
(g) from about 3% to about 16% by weight of water.

81. A process according to claim 35 wherein the fatty acid is a coconut fatty acid or a coconut fatty acid and stearic acid mixture.
